SEQUENCE LISTING (113 sequences)

SEQ ID NO: 1-3 - skipped (3 entries)

SEQ ID NO: 4 - DNA, 27 bp, synthetic construct
  source 1..27: mol_type = other DNA
gcattttttacattttttacatttttt

SEQ ID NO: 5-29 - skipped (25 entries)

SEQ ID NO: 30 - DNA, 14 bp, synthetic construct
  source 1..14: mol_type = other DNA
gatccgaaaacata

SEQ ID NO: 31 - skipped

SEQ ID NO: 32 - DNA, 14 bp, synthetic construct
  source 1..14: mol_type = other DNA
gatccgaaaacata

SEQ ID NO: 33 - DNA, 10 bp, synthetic construct
  source 1..10: mol_type = other DNA
ctaggctttt

SEQ ID NO: 34 - DNA, 14 bp, synthetic construct
  source 1..14: mol_type = other DNA
gatccgaaaacata

SEQ ID NO: 35 - skipped

SEQ ID NO: 36 - DNA, 14 bp, synthetic construct
  source 1..14: mol_type = other DNA
gtgtgcatgtgtag

SEQ ID NO: 37-53 - skipped (17 entries)

SEQ ID NO: 54 - DNA, 32 bp, synthetic construct
  source 1..32: mol_type = other DNA
attggttaagaattacctcatcccgaggcggc

SEQ ID NO: 55 - DNA, 22 bp, synthetic construct
  source 1..22: mol_type = other DNA
ccacaaacgcatatttcgtgtc

SEQ ID NO: 56 - DNA, 13 bp, synthetic construct
  source 1..13: mol_type = other DNA
ggtgtttgcgtat

SEQ ID NO: 57 - DNA, 22 bp, synthetic construct
  source 1..22: mol_type = other DNA
ccacaaacgcatatttcgtgtc

SEQ ID NO: 58 - DNA, 13 bp, synthetic construct
  source 1..13: mol_type = other DNA
ggtgtttgcgtat

SEQ ID NO: 59 - DNA, 22 bp, synthetic construct
  source 1..22: mol_type = other DNA
ccacaaacgcatatttcgtgtc

SEQ ID NO: 60 - DNA, 13 bp, synthetic construct
  source 1..13: mol_type = other DNA
ggtgtttgcgtat

SEQ ID NO: 61 - DNA, 22 bp, synthetic construct
  source 1..22: mol_type = other DNA
ccacaaacgcatatttcgtgtc

SEQ ID NO: 62 - DNA, 13 bp, synthetic construct
  source 1..13: mol_type = other DNA
ggtgtttgcgtat

SEQ ID NO: 63 - DNA, 22 bp, synthetic construct
  source 1..22: mol_type = other DNA
ccacaaacgcatatttcgtgtc

SEQ ID NO: 64 - DNA, 14 bp, synthetic construct
  source 1..14: mol_type = other DNA
ggtgtttgcgtata

SEQ ID NO: 65 - DNA, 20 bp, synthetic construct
  source 1..20: mol_type = other DNA
ccctgattatgattatacat

SEQ ID NO: 66 - DNA, 20 bp, synthetic construct
  source 1..20: mol_type = other DNA
ccctgattatgattatacat

SEQ ID NO: 67 - DNA, 10 bp, synthetic construct
  source 1..10: mol_type = other DNA
tccatcgact

SEQ ID NO: 68 - DNA, 10 bp, synthetic construct
  source 1..10: mol_type = other DNA
tctatcgact

SEQ ID NO: 69 - DNA, 10 bp, synthetic construct
  source 1..10: mol_type = other DNA
catatctata

SEQ ID NO: 70 - DNA, 10 bp, synthetic construct
  source 1..10: mol_type = other DNA
catatctatc

SEQ ID NO: 71-113 - skipped (43 entries)

(19) United States
(12) Patent Application Publication
(10) Pub. No.: US 2025/0215487 A1
(43) Pub. Date: Jul. 3, 2025

(54) NANOPORE SEQUENCING

(71) Applicant: Illumina, Inc., San Diego, CA (US)

(72) Inventors: Jeffrey Mandell, Rancho Santa Fe, CA (US); Jessica Killian, San Diego, CA (US)

(21) Appl. No.: 17/933,833

(22) Filed: Sep. 20, 2022

Related U.S. Application Data
(60) Provisional application No. 63/247,155, filed on Sep. 22, 2021.

(51) Int. Cl. C12Q 1/6869; C12Q 1/70; G01N 27/327; G01N 27/447

(52) U.S. Cl. CPC C12Q 1/6869; C12Q 1/701; G01N 27/3276; G01N 27/44791

(57) ABSTRACT

Systems and methods for sequencing polynucleotides using nanopores are disclosed. In some embodiments, a polynucleotide including a single-stranded region and a double-stranded region, in which the single-stranded region is disposed through a nanopore. The polynucleotide can be moved relative to the nanopore by electric forces while one or more structural locks keep the polynucleotide close to the nanopore. A characteristic signal based on nanopore ionic current blockade and associated with the regions of the polynucleotide at or near the nanopore recognition zone is measured and used to infer the nucleobase sequence of the polynucleotide. In some examples, the double-stranded region is extended by a polymerase, and the polymerase is removed from the polynucleotide. In some examples, signals measured under different applied voltages provide nonredundant information regarding the polynucleotide sequence.

### CROSS-REFERENCE TO RELATED APPLICATIONS

This application claims priority to U.S. Provisional Application No. 63/247,155, filed on Sep. 22, 2021, the content of which is incorporated by reference in its entirety.

### REFERENCE TO SEQUENCE LISTING

The present application is being filed along with a Sequence Listing in electronic format. The Sequence Listing is provided as an XML file entitled “ILLINC.700WO ST.26 Sequence Listing”, created and last saved on Nov. 22, 2022, which is 38,850 bytes in size, and further updated by a file entitled “20230406SequenceListingILLINC700A.XML” which was created on Apr. 6, 2023, and is approximately 39,080 bytes in size. The information in the electronic format of the Sequence Listing is hereby incorporated by reference in its entirety.

### BACKGROUND

Some DNA sequencing techniques involve performing a large number of controlled reactions on support surfaces or within predefined reaction chambers. The controlled reactions may then be observed or detected, and subsequent analysis may help identify properties of the polynucleotide involved in the reaction. Examples of such sequencing techniques include next-generation sequencing or massive parallel sequencing involving sequencing-by-ligation, sequencing-by-synthesis, reversible terminator chemistry, or pyrosequencing approaches.

Some DNA sequencing techniques can achieve single molecule resolution by utilizing a nanopore. For example, a nanopore disposed in a membrane provides a path for ionic current. As a single-stranded DNA (ssDNA) subject to an electrical driving force traverses through the nanopore, the ssDNA influences the ionic current through the nanopore. For example, each passing nucleotide of the single ssDNA or each series of nucleotides of the ssDNA that passes through the nanopore yields a characteristic ionic electrical current. Thus, signals (such as these characteristic electrical currents through the nanopore) associated with the traversing ssDNA can be recorded and be used to determine the sequence of the ssDNA. For example, the use of processive enzymes such as polymerases or helicases to aid the translocation of polynucleotides through nanopores may suffer from the stochastic behaviors of these enzymes and may thus have a highly variable translocation time per nucleotide. Further, even with the processive enzymes, the translocation speed may still be faster than the rate that can be accurately measured with electronics and detectors. However, previously known compositions, systems, and methods for nanopore sequencing may not be sufficiently robust, reproducible, accurate or sensitive, and may not have sufficiently high throughput or low cost for practical implementation. Accordingly, there remains a need for improved compositions, systems, and methods for nanopore sequencing.

Olasagasti et al., “Replication of individual DNA molecules under electronic control using a protein nanopore,” Nature Nanotechnology 5(11): 798-806 (2010) discloses disposing a DNA template through a nanopore. A DNA template-polymerase complex is formed on the first side of an α-hemolysin nanopore, and includes a DNA duplex and a polymerase. The DNA template includes abasic reporter nucleotides that initially are positioned on the second side of the α-hemolysin nanopore. While the polymerase is used to add nucleotides to the duplex based on the sequence of the DNA template, the ionic current through the nanopore is measured (I_{EBS}, where EBS refers to the enzyme bound state). As these nucleotides are added, the abasic reporter nucleotides are drawn towards and subsequently through the α-hemolysin, which causes changes in I_{EBS}.

### SUMMARY

In order to improve the confidence or accuracy of polynucleotide sequence identification, it may be desirable to have a controlled translocation process. In certain embodiments, a polymerase (e.g., DNA polymerase or reverse transcriptase) is utilized to incorporate nucleotides to synthesize a complementary strand on a template polynucleotide strand (e.g., single-stranded DNA or single-stranded RNA) in which the polymerase is removed after each additional nucleotide has been incorporated by pulling the template strand-complementary strand complex against the nanopore by electric forces. In certain embodiments, one or more structural “lock” moieties is utilized to help keep a template strand-complementary strand complex close to a nanopore while the complex is subject to electric forces. In certain embodiments, a region of the template strand-complementary strand complex close to a nanopore may be measured for an amount of time sufficient to achieve the desired signal-to-noise ratio. In certain embodiments, one or more structural “lock” moieties is utilized to isolate a region of a template strand for sequencing through a nanopore.

In certain embodiments, the same template polynucleotide molecule is sequenced more than once in the same nanopore or the same nanopore unit cell to improve consensus accuracy. For example, while nanopore sequencing of a template polynucleotide strand has been performed once as its complementary strand is being synthesized, the same template strand is sequenced an additional time in the same nanopore after stripping/removing the complementary strand from the template strand by pulling the template strand-complementary strand complex against the nanopore by electric forces, and this process may be repeated several rounds.

In certain embodiments, an average value of the signal (e.g., characteristic ionic electrical current through the nanopore) associated with the traversing polynucleotide and/or the information in the noise (e.g., variation or standard deviation) are utilized to identify one or more nucleotides in the polynucleotide. For example, the current through the nanopore may be modulated by the identities of one or more nucleotides near the nanopore. The identity of a nucleotide relates to whether a nucleotide has a base A, T, C, or G, or a non-natural base, or has other DNA or RNA modifications (e.g., epigenetic modifications or damage). Exemplary DNA and RNA modifications or non-natural bases include: methylated bases such as 5-methylcytosine (5-MeC) or N⁶-methyladenosine (N⁶-MeA), hydroxymethylated bases such as 5-hydroxymethylcytosine, pseudouridine bases, 7,8-dihydro-8-oxoguanine bases, 2′-O-methyl derivative bases, 4-thiouridine (s4U), 6-thioguanine (s6G), apurinic sites, apyrimidic sites, pyrimidine dimers, thymine dimers, DNA adducts, hydrolysis damage, oxidation damage, 8-oxoguanosine (8-oxoG), ribonucleosides within DNA, glucose-modified 5-hydroxymethylcytosine, HOMedU, β-D-glucosyl-HOMedU, cytosine-5-methylenesulfonate (CMS), etc. Additionally or alternatively, a waiting time before an additional nucleotide is successfully incorporated is utilized to infer information regarding the base in the template strand. For example, without being bound by theory, non-natural bases or modifications to the bases in the template strand may affect the rate of incorporating an additional nucleotide to the complementary strand, e.g., as the base-paring interactions may be affected.

In certain embodiments, various combinations of a number of bases in the single-stranded region and a number of bases in the double-stranded region of the polynucleotide being sensed at (or interacting with) the nanopore is utilized to identify one or more nucleotides. In certain embodiments, a combination that increases the sequence identification confidence or accuracy and use such combination in the process of inferring sequencing results in subsequent measurements.

In some embodiments, a combination and the associated signals to construct a “K-mer map” maps each instance of a K-mer polynucleotide sequence to a unique code, which may include a unique D-dimensional value extracted from a nanopore signal measured with the K-mer polynucleotide instance. In some examples, the K-mer map may be predictive (up to a predetermined level of discrepancy) of the series of codes associated with previously unmeasured polynucleotide sequences. In some examples, the K-mer map may allow for de novo sequencing of polynucleotides without the aid of a reference sequence (e.g., reference genome or transcriptome).

In some embodiments, without being bound by theory, several different magnitudes of an applied voltage are utilized to modulate a relative position and/or interactions between a polynucleotide and a nanopore, thus providing several interrogations of the incorporated nucleotide plus the neighboring nucleotides as a whole. In some examples, nonredundant, uncorrelated or independent pieces of information may be obtained through the several interrogations of the incorporated nucleotide plus the neighboring nucleotides as a whole under different applied voltages.

In some embodiments, certain embodiments of sequencing double-stranded polynucleotides using nanopores and/or certain embodiments of analyzing or organizing signals associated with K-mer polynucleotides can be utilized with the systems disclosed in U.S. provisional patent application Ser. No. 63/247,155 filed on Sep. 22, 2021 and U.S. patent application Ser. No. 17/224,496 published as US2021/0313009 on Oct. 7, 2021, the disclosures of which are incorporated herein by reference in their entireties.

Embodiment described herein may also include filtering of the signal level, such as filtering the signal level for an expected range of values. Although embodiments herein describe determining a signal level, such as mean current, over a whole time duration in a read position, embodiments also include alone or in combination determining a signal level for any portion of a duration in a read position, such as sampling of a signal one or more times during a read position. Although embodiments herein describe determining a signal level by using a mean value, embodiments also include alone or in combination determining a signal level by any statistical method, such as median, mode, slope, inflection, or other statistical method. Although embodiments herein describe determining a signal level by determining the ionic current through the nanopore, embodiments also include alone or in combination determining the signal level by measuring other electrical characteristics of the cis/trans nanopore cell. For example, in other embodiments, a signal level is determined by the voltage potential at a specified area or component of the cis/trans nanopore cell. For example, in other embodiments, a signal level is determined by the electrical impedance at a specified area or component of the cis/trans nanopore cell. For example, in other embodiments, a signal level is determined by the conductivity/resistance of the nanopore membrane.

The systems, devices, kits, and methods disclosed herein each have several aspects, no single one of which is solely responsible for their desirable attributes. Without limiting the scope of the claims, some prominent features will now be discussed briefly. Numerous other examples are also contemplated, including examples that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the devices and methods disclosed herein provide advantages over other known devices and methods.

It is to be understood that any features of the device and/or of the array disclosed herein may be combined together in any desirable manner and/or configuration. Further, it is to be understood that any features of the method of using the device may be combined together in any desirable manner. Moreover, it is to be understood that any combination of features of this method and/or of the device and/or of the array may be used together, and/or may be combined with any of the examples disclosed herein. Still further, it is to be understood that any feature or combination of features of any of the devices and/or of the arrays and/or of any of the methods may be combined together in any desirable manner, and/or may be combined with any of the examples disclosed herein.

It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A illustrates an example nanopore sequencing system which may be used to implement some embodiments of the disclosed technology.

FIG. 1B illustrates a portion of one of the nanopore unit cells of the example nanopore sequencing system of FIG. 1A.

FIG. 2A illustrates example sequencing operations used in some embodiments of the disclosed technology.

FIG. 2B shows an experimental result measured according to the example sequencing operations illustrated in FIG. 2A.

FIG. 3 illustrates a method for tailored sequencing according to some embodiments of the disclosed technology.

FIG. 4A and FIG. 4B illustrate another method for sequencing a polynucleotide according to some embodiments of scanning the template polynucleotide at more than one read potential consistent with the experiment associated with FIG. 2B.

FIG. 5, FIG. 6, FIG. 7A and FIG. 7B illustrate some embodiments of the disclosed technology involving re-sequencing or consensus sequencing of a template polynucleotide in order to sequence the polynucleotide more than once.

FIG. 8A illustrates a method of constructing and using a K-mer map.

FIG. 8B illustrates example K-mer instances and K-mer map constructions discussed in connection with FIG. 8A.

FIG. 9A illustrates a method of constructing and using a K-mer map for instances of K-mer including methylated C bases.

FIG. 9B illustrates example K-mer instances and K-mer map constructions discussed in connection with FIG. 9A.

FIG. 10A illustrates another method of constructing and using a K-mer map for instances of K-mer including methylated C bases, where the base incorporation waiting time may be utilized.

FIG. 10B shows experimental data relating to detecting epigenetic modifications via indirect kinetics, according to some embodiments of the disclosed technology.

FIG. 11A and FIG. 11B illustrate an example of making a K-mer map having 5-mers.

FIG. 12 illustrates examples of K-mer map states.

FIG. 13 illustrates an example of de novo sequencing of polynucleotides according to some embodiments of the disclosed technology.

FIG. 14A, FIG. 14B and FIG. 14C illustrate another example of de novo sequencing of polynucleotides according to some embodiments of the disclosed technology.

FIG. 15A, FIG. 15B and FIG. 15C show an example of using the noise of nanopore signals.

FIG. 16A, FIG. 16B, FIG. 16C, FIG. 16D, FIG. 17A and FIG. 17B illustrate examples of voltage-dependent modulation of the polynucleotide-nanopore interaction and non-Ohmic behavior of the ionic current.

FIG. 18-I, FIG. 18-II and FIG. 18-III show results of de novo sequencing of the PhiX bacteriophage genome.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The terms comprising, including, containing and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

As used herein, the term “membrane” refers to a non-permeable or semi-permeable barrier or other sheet that separates two liquid/gel chambers (e.g., a cis well and a fluidic cavity or reservoir) which can contain the same compositions or different compositions therein. The permeability of the membrane to any given species depends upon the nature of the membrane. In some examples, the membrane may be non-permeable to ions, to electric current, and/or to fluids. For example, a lipid membrane may be impermeable to ions (i.e., does not allow any ion transport therethrough), but may be at least partially permeable to water (e.g., water diffusivity ranges from about 40 μm/s to about 100 μm/s). For another example, a synthetic/solid-state membrane, one example of which is silicon nitride, may be impermeable to ions, electric charge, and fluids (i.e., the diffusion of all of these species is zero). Any membrane may be used in accordance with the present disclosure, as long as the membrane can include a transmembrane nanoscale opening and can maintain a potential difference across the membrane. The membrane may be a monolayer or a multilayer membrane. A multilayer membrane includes two or more layers, each of which is a non-permeable or semi-permeable material.

The membrane may be formed of materials of biological or non-biological origin. A material that is of biological origin refers to material derived from or isolated from a biological environment such as an organism or cell, or a synthetically manufactured version of a biologically available structure (e.g., a biomimetic material).

An example membrane that is made from the material of biological origin includes a monolayer formed by a bolalipid. Another example membrane that is made from the material of biological origin includes a lipid bilayer. Suitable lipid bilayers include, for example, a membrane of a cell, a membrane of an organelle, a liposome, a planar lipid bilayer, and a supported lipid bilayer. A lipid bilayer can be formed, for example, from two opposing layers of phospholipids, which are arranged such that their hydrophobic tail groups face towards each other to form a hydrophobic interior, whereas the hydrophilic head groups of the lipids face outwards towards the aqueous environment on each side of the bilayer. Lipid bilayers also can be formed, for example, by a method in which a lipid monolayer is carried on an aqueous solution/air interface past either side of an aperture that is substantially perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has at least partially evaporated, the solution/air interfaces on either side of the aperture are physically moved up and down past the aperture until a bilayer is formed. Other suitable methods of bilayer formation include tip-dipping, painting bilayers, and patch-clamping of liposome bilayers. Any other methods for obtaining or generating lipid bilayers may also be used.

A material that is not of biological origin may also be used as the membrane. Some of these materials are solid-state materials and can form a solid-state membrane, and others of these materials can form a thin liquid film or membrane. The solid-state membrane can be a monolayer, such as a coating or film on a supporting substrate (i.e., a solid support), or a freestanding element. The solid-state membrane can also be a composite of multilayered materials in a sandwich configuration. Any material not of biological origin may be used, as long as the resulting membrane can include a transmembrane nanoscale opening and can maintain a potential difference across the membrane. The membranes may include organic materials, inorganic materials, or both. Examples of suitable solid-state materials include, for example, microelectronic materials, insulating materials (e.g., silicon nitride (Si₃N₄), aluminum oxide (Al₂O₃), hafnium oxide (HfO₂), tantalum pentoxide (Ta₂O₅), silicon oxide (SiO₂), etc.), some organic and inorganic polymers (e.g., polyamide, plastics, such as polytetrafluoroethylene (PTFE), or elastomers, such as two-component addition-cure silicone rubber), and glasses. In addition, the solid-state membrane can be made from a monolayer of graphene, which is an atomically thin sheet of carbon atoms densely packed into a two-dimensional honeycomb lattice, a multilayer of graphene, or one or more layers of graphene mixed with one or more layers of other solid-state materials. A graphene-containing solid-state membrane can include at least one graphene layer that is a graphene nanoribbon or graphene nanogap, which can be used as an electrical sensor to characterize the target polynucleotide. It is to be understood that the solid-state membrane can be made by any suitable method, for example, chemical vapor deposition (CVD). In an example, a graphene membrane can be prepared through either CVD or exfoliation from graphite. Examples of suitable thin liquid film materials that may be used include diblock copolymers or triblock copolymers, such as amphiphilic PMOXA-PDMS-PMOXA ABA triblock copolymers.

The application of an electric potential difference across a nanopore may bias the translocation of a nucleic acid relative to the nanopore. One or more signals are generated that correspond to the translocation of the nucleotide through the nanopore. Accordingly, as a target polynucleotide, or as a mononucleotide or a probe derived from the target polynucleotide or mononucleotide, transits through the nanopore, the current across the membrane changes due to base-dependent (or probe dependent) blockage of the constriction, for example. The signal from that change in current can be measured using any of a variety of methods. Each signal is unique to the species of nucleotide(s) (or probe or linker constructs with a reporter barcode region) in the nanopore, such that the resultant signal can be used to determine a characteristic of the polynucleotide. For example, the identity of one or more species of nucleotide(s) (or probe) that produces a characteristic signal can be determined.

As used herein, a “reporter” is composed of one or more reporter elements. Reporters include what are known as “tags” and “labels.” Reporters serve to parse the genetic information of the target nucleic acid. “Encode” or “parse” are verbs referring to transferring from one format to another, and refers to transferring the genetic information of target template base sequence into an arrangement of reporters.

As used herein, the term “nanopore” is intended to mean a hollow structure discrete from, or defined in, and extending across the membrane. The nanopore permits ions, electric current, and/or fluids to cross from one side of the membrane to the other side of the membrane. For example, a membrane that inhibits the passage of ions or water-soluble molecules can include a nanopore structure that extends across the membrane to permit the passage (through a nanoscale opening extending through the nanopore structure) of the ions or water-soluble molecules from one side of the membrane to the other side of the membrane. The diameter of the nanoscale opening extending through the nanopore structure can vary along its length (i.e., from one side of the membrane to the other side of the membrane), but at any point is on the nanoscale (i.e., from about 1 nm to about 100 nm, or to less than 1000 nm). Examples of the nanopore include, for example, biological nanopores, solid-state nanopores, and biological and solid-state hybrid nanopores.

As used herein, the term “diameter” is intended to mean the longest straight line inscribable in a cross-section of a nanoscale opening through a centroid of the cross-section of the nanoscale opening. It is to be understood that the nanoscale opening may or may not have a circular or substantially circular cross-section (the cross-section of the nanoscale opening being substantially parallel with the cis/trans electrodes). Further, the cross-section may be regularly or irregularly shaped.

As used herein, the term “biological nanopore” is intended to mean a nanopore whose structure portion is made from materials of biological origin. Biological origin refers to a material derived from or isolated from a biological environment such as an organism or cell, or a synthetically manufactured version of a biologically available structure. Biological nanopores include, for example, polypeptide nanopores and polynucleotide nanopores.

As used herein, the term “polypeptide nanopore” is intended to mean a protein/polypeptide that extends across the membrane, and permits ions, electric current, biopolymers such as DNA or peptides, or other molecules of appropriate dimension and charge, and/or fluids to flow therethrough from one side of the membrane to the other side of the membrane. A polypeptide nanopore can be a monomer, a homopolymer, or a heteropolymer. Structures of polypeptide nanopores include, for example, an α-helix bundle nanopore and a β-barrel nanopore. Example polypeptide nanopores include α-hemolysin, Mycobacterium smegmatis porin A (MspA), gramicidin A, maltoporin, OmpF, OmpC, PhoE, Tsx, F-pilus, aerolysin, etc. The protein α-hemolysin is found naturally in cell membranes, where it acts as a pore for ions or molecules to be transported in and out of cells. Mycobacterium smegmatis porin A (MspA) is a membrane porin produced by Mycobacteria, which allows hydrophilic molecules to enter the bacterium. MspA forms a tightly interconnected octamer and transmembrane beta-barrel that resembles a goblet and contains a central pore.

A polypeptide nanopore can be synthetic. A synthetic polypeptide nanopore includes a protein-like amino acid sequence that does not occur in nature. The protein-like amino acid sequence may include some of the amino acids that are known to exist but do not form the basis of proteins (i.e., non-proteinogenic amino acids). The protein-like amino acid sequence may be artificially synthesized rather than expressed in an organism and then purified/isolated.

As used herein, the term “polynucleotide nanopore” is intended to include a polynucleotide that extends across the membrane, and permits ions, electric current, and/or fluids to flow from one side of the membrane to the other side of the membrane. A polynucleotide pore can include, for example, a polynucleotide origami (e.g., nanoscale folding of DNA to create the nanopore).

Also as used herein, the term “solid-state nanopore” is intended to mean a nanopore whose structure portion is defined by a solid-state membrane and includes materials of non-biological origin (i.e., not of biological origin). A solid-state nanopore can be formed of an inorganic or organic material. Solid-state nanopores include, for example, silicon nitride nanopores, silicon dioxide nanopores, and graphene nanopores.

The nanopores disclosed herein may be hybrid nanopores. A “hybrid nanopore” refers to a nanopore including materials of both biological and non-biological origins. An example of a hybrid nanopore includes a polypeptide-solid-state hybrid nanopore and a polynucleotide-solid-state nanopore.

In some embodiments, the nanopore may comprise a solid-state material, such as silicon nitride, modified silicon nitride, silicon, silicon oxide, or graphene, or a combination thereof. In some embodiments, the nanopore is a protein that forms a tunnel upon insertion into a bilayer, membrane, thin film, or solid-state aperture. In some embodiments, the nanopore is comprised in a lipid bilayer. In some embodiments, the nanopore is comprised in an artificial membrane comprising a mycolic acid. The nanopore may be a Mycobacterium smegmatis porin (Msp) having a vestibule and a constriction zone that define the tunnel. The Msp porin may be a mutant MspA porin. In some embodiments, amino acids at positions 90, 91, and 93 of the mutant MspA porin are each substituted with asparagine. Some embodiments may comprise altering the translocation velocity or sequencing sensitivity by removing, adding, or replacing at least one amino acid of an Msp porin. A “mutant MspA porin” is a multimer complex that has at least or at most 70, 75, 80, 85, 90, 95, 98, or 99 percent or more identity, or any range derivable therein, but less than 100%, to its corresponding wild-type MspA porin and retains tunnel-forming capability. A mutant MspA porin may be recombinant protein. Optionally, a mutant MspA porin is one having a mutation in the constriction zone or the vestibule of a wild-type MspA porin. Optionally, a mutation may occur in the rim or the outside of the periplasmic loops of a wild-type MspA porin. A mutant MspA porin may be employed in any embodiment described herein.

A “vestibule” refers to the cone-shaped portion of the interior of an Msp porin whose diameter generally decreases from one end to the other along a central axis, where the narrowest portion of the vestibule is connected to the constriction zone. A vestibule may also be referred to as a “goblet.” The vestibule and the constriction zone together define the tunnel of an Msp porin. A “constriction zone” or the “readhead” refers to the narrowest portion of the tunnel of an Msp porin, in terms of diameter, that is connected to the vestibule. The length of the constriction zone may range from about 0.3 nm to about 2 nm. Optionally, the length is about, at most about, or at least about 0.3, 0.4, 0.5, 0.6, 0.7, 0.8, 0.9, 1.0, 1.1, 1.2, 1.3, 1.4, 1.5, 1.6, 1.7, 1.8, 1.9, 2, or 3 nm, or any range derivable therein. The diameter of the constriction zone may range from about 0.3 nm to about 2 nm. Optionally, the diameter is about, at most about, or at least about 0.3, 0.4, 0.5, 0.6, 0.7, 0.8, 0.9, 1.0, 1.1, 1.2, 1.3, 1.4, 1.5, 1.6, 1.7, 1.8, 1.9, 2, or 3 nm, or any range derivable therein. A “tunnel” refers to the central, empty portion of an Msp porin that is defined by the vestibule and the constriction zone, through which a gas, liquid, ion, or analyte may pass. A tunnel is an example of an opening of a nanopore.

Various conditions such as light and the liquid medium that contacts a nanopore, including its pH, buffer composition, detergent composition, and temperature, may affect the behavior of the nanopore, particularly with respect to its conductance through the tunnel as well as the movement of an analyte with respect to the tunnel, either temporarily or permanently.

In some embodiments, the disclosed system for nanopore sequencing comprises an Msp porin having a vestibule and a constriction zone that define a tunnel, wherein the tunnel is positioned between a first liquid medium and a second liquid medium, wherein at least one liquid medium comprises an analyte polynucleotide, and wherein the system is operative to detect a property of the analyte. The system may be operative to detect a property of any analyte comprising subjecting an Msp porin to an electric field such that the analyte interacts with the Msp porin. The system may be operative to detect a property of the analyte comprising subjecting the Msp porin to an electric field such that the analyte electrophoretically translocates relative to the tunnel of the Msp porin. In some embodiments, the system comprises an Msp porin having a vestibule and a constriction zone that define a tunnel, wherein the tunnel is positioned in a lipid bilayer between a first liquid medium and a second liquid medium, and wherein the only point of liquid communication between the first and second liquid media occurs in the tunnel. Moreover, any Msp porin described herein may be comprised in any system described herein.

The system may further comprise one or more temperature regulating devices in communication with the fluid or electrolyte. The system described herein may be operative to translocate an analyte through an Msp porin tunnel either electrophoretically or otherwise.

As used herein, a “peptide” refers to two or more amino acids joined together by an amide bond (that is, a “peptide bond”). Peptides comprise up to or include 50 amino acids. Peptides may be linear or cyclic. Peptides may be α, β, γ, δ, or higher, or mixed. Peptides may comprise any mixture of amino acids as defined herein, such as comprising any combination of D, L, α, β, γ, δ, or higher amino acids.

As used herein, a “protein” refers to an amino acid sequence having 51 or more amino acids.

As used herein, a “polymerase” is an enzyme generally used for joining 3′-OH 5′-triphosphate nucleotides, oligomers, and their analogs. Polymerases include, but are not limited to, Bsu DNA Polymerase, IsoPol™ DNA Polymerase (ArcticZymes Technologies ASA), DNA-dependent DNA polymerases, DNA-dependent RNA polymerases, RNA-dependent DNA polymerases, RNA-dependent RNA polymerases, T7 DNA polymerase, T3 DNA polymerase, T4 DNA polymerase, T7 RNA polymerase, T3 RNA polymerase, SP6 RNA polymerase, DNA polymerase I, Klenow fragment, Thermophilus aquaticus DNA polymerase, Tth DNA polymerase, VentR® DNA polymerase (New England Biolabs), Deep VentR® DNA polymerase (New England Biolabs), Bst DNA Polymerase Large Fragment, Stoeffel Fragment, 90N DNA Polymerase, 90N DNA polymerase, Pfu DNA Polymerase, TfI DNA Polymerase, Tth DNA Polymerase, RepliPHI Phi29 Polymerase, TIi DNA polymerase, eukaryotic DNA polymerase beta, telomerase, Therminator™ polymerase (New England Biolabs), KOD HiFi™ DNA polymerase (Novagen), KOD1 DNA polymerase, Q-beta replicase, terminal transferase, AMV reverse transcriptase, M-MLV reverse transcriptase, Phi6 reverse transcriptase, HIV-1 reverse transcriptase, novel polymerases discovered by bioprospecting, and polymerases cited in US 2007/0048748, U.S. Pat. Nos. 6,329,178, 6,602,695, and U.S. Pat. No. 6,395,524 (incorporated by reference). These polymerases include wild-type, mutant isoforms, and genetically engineered variants.

As used herein, “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties.

As used herein, the term “nucleotide” is intended to mean a molecule that includes a sugar and at least one phosphate group, and in some examples also includes a nucleobase. A nucleotide that lacks a nucleobase may be referred to as “abasic.” In some embodiments, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides are natural nucleotides, but it is to be further understood that non-natural nucleotides, modified nucleotides or analogs of the aforementioned nucleotides can also be used.

Examples of nucleotides may include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

Examples of nucleotides may also be intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

In some embodiments, the term “modification” as used herein is intended to refer not only to a chemical modification of a nucleic acids, but also to a variation in nucleic acid conformation or composition, interaction of an agent with a nucleic acid (e.g., bound to the nucleic acid), and other perturbations associated with the nucleic acid. As such, a location or position of a modification is a locus (e.g., a single nucleotide or multiple contiguous or noncontiguous nucleotides) at which such modification occurs within the nucleic acid. For a double-stranded template, such a modification may occur in the strand complementary to a nascent strand synthesized by a polymerase processing the template, or may occur in the displaced strand. For example, modified nucleotides may include 5-methylcytosine, N6-methyladenosine, N3-methyladenosine, N7-methylguanosine, 5-hydroxymethylcytosine, pseudouridine, thiouridine, isoguanosine, isocytosine, dihydrouridine, queuosine, wyosine, inosine, triazole, diaminopurine, β-D-glucopyranosyloxymethyluracil (a.k.a., β-D-glucosyl-HOMedU, β-glucosyl-hydroxymethyluracil, “dJ,” or “base J”), 8-oxoguanosine, and 2′-O-methyl derivatives of adenosine, cytidine, guanosine, and uridine. Modified DNA and RNA bases are further described, for example, in Narayan P, et al. (1987) Mol Cell Biol 7(4):1572-5; Horowitz S, et al. (1984) Proc Natl Acad Sci U.S.A. 81(18):5667-71; “RNA's Outfits: The nucleic acid has dozens of chemical costumes,” (2009) C&EN; 87(36):65-68; Kriaucionis, et al. (2009) Science 324 (5929): 929-30; and Tahiliani, et al. (2009) Science 324 (5929): 930-35; Matray, et al. (1999) Nature 399(6737):704-8; Ooi, et al. (2008) Cell 133: 1145-8; Petersson, et al. (2005) J Am Chem Soc. 127(5):1424-30; Johnson, et al. (2004) 32(6):1937-41; Kimoto, et al. (2007) Nucleic Acids Res. 35(16):5360-9; Ahle, et al. (2005) Nucleic Acids Res 33(10):3176; Krueger, et al., Curr Opinions in Chem Biology 2007, 11(6):588); Krueger, et al. (2009) Chemistry & Biology 16(3):242; McCullough, et al. (1999) Annual Rev of Biochem 68:255; Liu, et al. (2003) Science 302(5646):868-71; Limbach, et al. (1994) Nucl. Acids Res. 22(12):2183-2196; Wyatt, et al. (1953) Biochem. J. 55:774-782; Josse, et al. (1962) J. Biol. Chem. 237:1968-1976; Lariviere, et al. (2004) J. Biol. Chem. 279:34715-34720; and in International Application Publication No. WO/2009/037473, the disclosures of which are incorporated herein by reference in their entireties.

Modifications may further include the presence of non-natural base pairs in the nucleic acid, including but not limited to hydroxypyridone and pyridopurine homo- and hetero-base pairs, pyridine-2,6-dicarboxylate and pyridine metallo-base pairs, pyridine-2,6-dicarboxamide and a pyridine metallo-base pairs, metal-mediated pyrimidine base pairs T-Hg(II)-T and C—Ag(I)-C, and metallo-homo-basepairs of 2,6-bis(ethylthiomethyl)pyridine nucleobases Spy, and alkyne-, enamine-, alcohol-, imidazole-, guanidine-, and pyridyl-substitutions to the purine or pyridimine base (Wettig, et al. (2003) J Inorg Biochem 94:94-99; Clever, et al. (2005) Angew Chem Int Ed 117:7370-7374; Schlegel, et al. (2009) Org Biomol Chem 7(3):476-82; Zimmerman, et al. (2004) Bioorg Chem 32(1):13-25; Yanagida, et al. (2007) Nucleic Acids Symp Ser (Oxf) 51:179-80; Zimmerman (2002) J Am Chem Soc 124(46):13684-5; Buncel, et al. (1985) Inorg Biochem 25:61-73; Ono, et al. (2004) Angew Chem 43:4300-4302; Lee, et al. (1993) Biochem Cell Biol 71:162-168; Loakes, et al. (2009), Chem Commun 4619-4631; and Seo, et al. (2009) J Am Chem Soc 131:3246-3252, the disclosures of which are incorporated herein by reference in their entireties). Other types of modifications include, e.g, a nick, a missing base (e.g., apurinic or apyridinic sites), a ribonucleoside (or modified ribonucleoside) within a deoxyribonucleoside-based nucleic acid, a deoxyribonucleoside (or modified deoxyribonucleoside) within a ribonucleoside-based nucleic acid, a pyrimidine dimer (e.g., thymine dimer or cyclobutane pyrimidine dimer), a cis-platin crosslinking, oxidation damage, hydrolysis damage, other methylated bases, bulky DNA or RNA base adducts, photochemistry reaction products, interstrand crosslinking products, mismatched bases, and other types of “damage” to the nucleic acid. Modified nucleotides can be caused by exposure of the DNA to radiation (e.g., UV), carcinogenic chemicals, crosslinking agents (e.g., formaldehyde), certain enzymes (e.g., nickases, glycosylases, exonucleases, methylases, other nucleases, glucosyltransferases, etc.), viruses, toxins and other chemicals, thermal disruptions, and the like.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

The terms “oligonucleotide” and “polynucleotide” may be used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

The term “nucleic acid” and “polynucleotide” may be used interchangeably to refer to a deoxyribonucleotide or ribonucleotide polymer in either single- or double-stranded form, and unless otherwise limited, encompasses known analogs of natural nucleotides that hybridize to nucleic acids in manner similar to naturally occurring nucleotides, such as peptide nucleic acids (PNAs) and phosphorothioate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

As used herein, the term “primer” is defined as a polynucleotide to which nucleotides may be added via a free 3′ OH group. A primer may include a 3′ block inhibiting polymerization until the block is removed. A primer may include a modification at the 5′ terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.”

As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either the 3′ end or the 5′ end the target polynucleotide. Target polynucleotides may be used without any adapter, in which case a primer binding sequence may come directly from a sequence found in the target polynucleotide.

For example, a template polynucleotide chain may be any sample that is to be sequenced, and may be composed of DNA, RNA, or analogs thereof (e.g., peptide nucleic acids). The source of the template (or target) polynucleotide chain can be genomic DNA, messenger RNA, or other nucleic acids from native sources. In some cases, the template polynucleotide chain that is derived from such sources can be amplified prior to use. Any of a variety of known amplification techniques can be used including, but not limited to, polymerase chain reaction (PCR), rolling circle amplification (RCA), multiple displacement amplification (MDA), or random primer amplification (RPA). It is to be understood that amplification of the template polynucleotide chain prior to use is optional. As such, the template polynucleotide chain will not be amplified prior to use in some examples. Template/target polynucleotide chains can optionally be derived from synthetic libraries. Synthetic nucleic acids can have native DNA or RNA compositions or can be analogs thereof.

Biological samples from which the template polynucleotide chain can be derived include, for example, those from a mammal, such as a rodent, mouse, rat, rabbit, guinea pig, ungulate, horse, sheep, pig, goat, cow, cat, dog, primate, human or non-human primate; a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii Takifugu rubripes, yeast, Saccharamoyces cerevisiae or Schizosaccharomyces pombe; or a Plasmodium falciparum. Template polynucleotide chains 48 can also be derived from prokaryotes such as a bacterium, Escherichia coli, staphylococci or Mycoplasma pneumoniae; an archae; a virus such as Hepatitis C virus, ebola virus or human immunodeficiency virus; or a viroid. Template polynucleotide chains can be derived from a homogeneous culture or population of the above organisms or alternatively from a collection of several different organisms, for example, in a community or ecosystem.

Moreover, template polynucleotide chains may not be derived from natural sources, but rather can be synthesized using known techniques. For example, gene expression probes or genotyping probes can be synthesized and used in the examples set forth herein.

In some examples, template polynucleotide chains can be obtained as fragments of one or more larger nucleic acids. Fragmentation can be carried out using any of a variety of techniques known in the art including, for example, nebulization, sonication, chemical cleavage, enzymatic cleavage, or physical shearing. Fragmentation may also result from use of a particular amplification technique that produces amplicons by copying only a portion of a larger nucleic acid chain. For example, PCR amplification produces fragments having a size defined by the length of the nucleotide sequence on the original template that is between the locations where flanking primers hybridize during amplification. The length of the template polynucleotide chain may be in terms of the number of nucleotides or in terms of a metric length (e.g., nanometers).

A population of template/target polynucleotide chains, or amplicons thereof, can have an average strand length that is desired or appropriate for a particular sequencing device. For example, the average strand length can be less than about 100,000 nucleotides, about 50,000 nucleotides, about 10,000 nucleotides, about 5,000 nucleotides, about 1,000 nucleotides, about 500 nucleotides, about 100 nucleotides, or about 50 nucleotides. Alternatively or additionally, the average strand length can be greater than about 10 nucleotides, about 50 nucleotides, about 100 nucleotides, about 500 nucleotides, about 1,000 nucleotides, about 5,000 nucleotides, about 10,000 nucleotides, about 50,000 nucleotides, or about 100,000 nucleotides. Alternatively or additionally, the average strand length can be greater than about 10 kilo nucleotides, about 50 kilo nucleotides, about 100 kilo nucleotides, about 500 kilo nucleotides, about 1,000 kilo nucleotides, about 5,000 kilo nucleotides, about 10,000 kilo nucleotides, about 50,000 kilo nucleotides, or about 100,000 kilo nucleotides. Alternatively or additionally, the average strand length can be greater than about 10 mega nucleotides, about 50 mega nucleotides, about 100 mega nucleotides, about 500 mega nucleotides, about 1,000 mega nucleotides, about 5,000 mega nucleotides, about 10,000 mega nucleotides, about 50,000 mega nucleotides, or about 100,000 mega nucleotides. The average strand length for a population of target polynucleotide chains, or amplicons thereof, can be in a range between a maximum and minimum value set forth above.

In some cases, a population of template/target polynucleotide chains can be produced under conditions or otherwise configured to have a maximum length for its members. For example, the maximum length for the members can be less than about 100,000 nucleotides, about 50,000 nucleotides, about 10,000 nucleotides, about 5,000 nucleotides, about 1,000 nucleotides, about 500 nucleotides, about 100 nucleotides or about 50 nucleotides. For example, the maximum length for the members can be less than about 100,000 kilo nucleotides, about 50,000 kilo nucleotides, about 10,000 kilo nucleotides, about 5,000 kilo nucleotides, about 1,000 kilo nucleotides, about 500 kilo nucleotides, about 100 kilo nucleotides or about 50 kilo nucleotides. For example, the maximum length for the members can be less than about 100,000 mega nucleotides, about 50,000 mega nucleotides, about 10,000 mega nucleotides, about 5,000 mega nucleotides, about 1,000 mega nucleotides, about 500 mega nucleotides, about 100 mega nucleotides or about 50 mega nucleotides. Alternatively or additionally, a population of template polynucleotide chains, or amplicons thereof, can be produced under conditions or otherwise configured to have a minimum length for its members. For example, the minimum length for the members can be more than about 10 nucleotides, about 50 nucleotides, about 100 nucleotides, about 500 nucleotides, about 1,000 nucleotides, about 5,000 nucleotides, about 10,000 nucleotides, about 50,000 nucleotides, or about 100,000 nucleotides. For example, the minimum length for the members can be more than about 10 kilo nucleotides, about 50 kilo nucleotides, about 100 kilo nucleotides, about 500 kilo nucleotides, about 1,000 kilo nucleotides, about 5,000 kilo nucleotides, about 10,000 kilo nucleotides, about 50,000 kilo nucleotides, or about 100,000 kilo nucleotides. For example, the minimum length for the members can be more than about 10 mega nucleotides, about 50 mega nucleotides, about 100 mega nucleotides, about 500 mega nucleotides, about 1,000 mega nucleotides, about 5,000 mega nucleotides, about 10,000 mega nucleotides, about 50,000 mega nucleotides, or about 100,000 mega nucleotides. The maximum and minimum strand length for template polynucleotide chains in a population can be in a range between a maximum and minimum value set forth above.

As used herein, the term “nanopore sequencer” refers to any of the devices disclosed herein that can be used for nanopore sequencing. In the examples disclosed herein, during nanopore sequencing, the nanopore is immersed in example(s) of the electrolyte disclosed herein and a potential difference is applied across the membrane. In an example, the potential difference is an electric potential difference or an electrochemical potential difference. An electric potential difference can be imposed across the membrane via a voltage source that injects or administers current to at least one of the ions of the electrolyte contained in the cis well or one or more of the trans wells. An electrochemical potential difference can be established by a difference in ionic composition of the cis and trans wells in combination with an electric potential. The different ionic composition can be, for example, different ions in each well or different concentrations of the same ions in each well.

The terms top, bottom, lower, upper, on, etc. are used herein to describe the device/nanopore sequencer and/or the various components of the device. It is to be understood that these directional terms are not meant to imply a specific orientation, but are used to designate relative orientation between components. The use of directional terms should not be interpreted to limit the examples disclosed herein to any specific orientation(s). As used herein, the terms “upper”, “lower”, “vertical”, “horizontal” and the like are meant to indicate relative orientation.

As used herein, the term “operably connected” refers to a configuration of elements, wherein an action or reaction of one element affects another element, but in a manner that preserves each element's functionality.

As used herein, the terms “fluidically connecting,” “fluid communication,” “fluidically coupled,” and the like refer to two spatial regions being connected together such that a fluid (e.g., liquid or gas) may flow between the two spatial regions. For example, a cis well/wells may be fluidically connected to a trans well/wells by way of a middle well and/or a nanochannel, such that a fluid, e.g., at least a portion of an electrolyte, may flow between the connected wells.

As used herein, the term “ionic connection” and the like refer to two spatial regions being connected together such that certain species of ions may flow between the two spatial regions.

As used herein, the term “electric connection” and the like refer to two spatial regions being connected together such that electrons, holes, ions or other charge carriers may flow between the two spatial regions.

If an electrolyte flows between two connected wells, ions and electric currents may also flow between the connected wells. In some examples, two spatial regions may be in fluid/ionic/electric communication through first and second nanoscale openings, or through one or more valves, restrictors, or other fluidic components that are to control or regulate a flow of fluid, ions or electric current through a system.

As used herein, the term “signal” is intended to mean an indicator that represents information. Signals include, for example, an electrical signal and an optical signal. The term “electrical signal” refers to an indicator of an electrical quality that represents information. The indicator can be, for example, current, voltage, tunneling, resistance, potential, voltage, conductance, or a transverse electrical effect (and any time-derivatives or transients of theses). An “electronic current” or “electric current” refers to a flow of electric charge. In an example, an electrical signal may be an electric current passing through a nanopore, and the electric current may flow when an electric potential difference is applied across the nanopore.

As used herein, the term “driving force” is intended to mean an electrical current that allows a polynucleotide to translocate relative the nanopore. In some embodiments, the electrical current electric current may flow when an electric potential difference is applied across the nanopore.

As used herein, “cis” refers to the area on one side of a membrane and “trans” refers to the other area on the other side of the membrane. “cis” generally refers to the side of a nanopore opening where the polymerase is located, usually where the polynucleotide template is initially introduced into the nanopore. “trans” generally refers to the side of a nanopore opening opposite to where the polymerase is located, usually the side through which an analyte or modified analyte (or fragments thereof) exits the opening. However, in some embodiments, an analyte can enter the nanopore through the “trans” side and exits through the “cis” side. “cis” and “trans” may also be used to refer to components or aspects of the system, such as a cis electrode, a trans electrode, a cis chamber, a trans chamber, etc.

As used herein, “translocation” and grammatical variants means that an analyte (e.g., DNA, reporter, tab, label, etc.) moves relatively to a nanopore. In some embodiments, an analyte may enter one side of an opening of a nanopore and move to and out of the other side of the opening. It is contemplated that any embodiment herein comprising translocation may refer to electrophoretic translocation or non-electrophoretic translocation, unless specifically noted. An electric field may move an analyte (e.g., a polynucleotide) or modified analyte relative to an opening of a nanopore. Optionally, methods that do not employ electrophoretic translocation are contemplated. In some embodiments, physical pressure causes a modified analyte to translocate relative to an opening of a nanopore. In some embodiments, a magnetic bead is attached to an analyte or modified analyte, and magnetic force causes the analyte or modified analyte to translocate relative to an opening of a nanopore. Other methods for translocation include but not limited to gravity, osmotic forces, temperature, and other physical forces such as centripetal force. In some embodiments, the analyte (e.g., DNA) or modified analyte may interact with the nanopore while translocating relative to the nanopore.

As used herein, the terms “well”, “cavity”, “reservoir” and “chamber” are used synonymously, and refer to a discrete feature defined in the device that can contain a fluid (e.g., liquid, gel, gas). A cis well is a chamber that contains or is partially defined by a cis electrode, and is also fluidically connected to a middle well where measurements occur (for example, by a FET, or by a metal electrode connected to an amplifier, a data acquisition device, or other signal conditioning elements such as analog filters, buffers, gain amplifiers, ADCs, etc.). The middle well in turn is fluidically connected to a trans well/chamber, in some examples. Examples of an array of the present device may have one cis well, for example one global cis chamber/reservoir, or multiple cis wells. The trans well is a single chamber that contains or is partially defined by its own trans electrode, and is also fluidically connected to a cis well. In examples including multiple trans wells, each trans well is electrically isolated from each other trans well. Further, it is to be understood that the cross-section of a well taken parallel to a surface of a substrate at least partially defining the well can be curved, square, polygonal, hyperbolic, conical, angular, etc.

As used herein, the term “electrode” is intended to mean a solid structure that conducts electricity. Electrodes may include any suitable electrically conductive material, such as gold, palladium, or platinum, or combinations thereof. In some examples, an electrode may be disposed on a substrate. In some examples, an electrode may define a substrate.

The term “substrate” refers to a rigid, solid support that is insoluble in aqueous liquid and is incapable of passing a liquid absent an aperture, port, or other like liquid conduit. In the examples disclosed herein, the substrate may have wells or chambers defined therein. Examples of suitable substrates include glass and modified or functionalized glass, plastics (including acrylics, polystyrene and copolymers of styrene and other materials, polypropylene, polyethylene, polybutylene, polyurethanes, polytetrafluoroethylene (PTFE) (such as TEFLON® from Chemours), cyclic olefins/cyclo-olefin polymers (COP) (such as ZEONOR® from Zeon), polyimides, etc.), nylon, ceramics, silica or silica-based materials, silicon and modified silicon, carbon, metals, inorganic glasses, and optical fiber bundles.

The aspects and examples set forth herein and recited in the claims can be understood in view of the above definitions.

### Nanopore Sequencing System

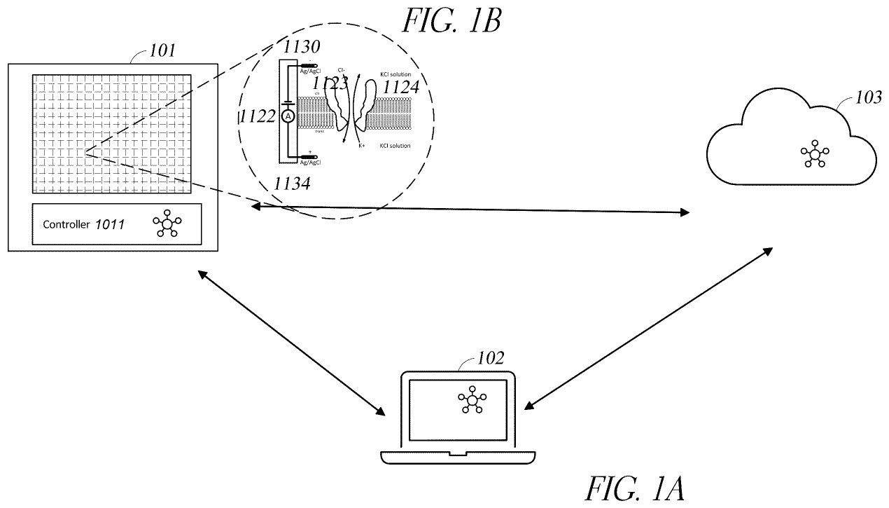

FIG. 1A illustrates an example nanopore sequencing system which may be used to implement some embodiments. The example nanopore sequencing system may include a nanopore sequencer 101, which may include a controller 1011 and an array of nanopore unit cells. The controller 1011 may be configured to control the sequencing operations in the array of nanopore unit cells. The example nanopore sequencing system may further include a computer 102 that is operably connected with the nanopore sequencer 101. The example nanopore sequencing system may further include a data storage and computing resource 103, such as a network or cloud, which may be operably connected with the nanopore sequencer 101 and the computer 102.

The recognition zone of a nanopore (such as a MspA) may include a constriction, which may be the most sensitive region for nucleotide discrimination since the constriction is where the largest voltage drop occurs as it presents the largest resistance between the cis and trans electrodes. Nanopore recognition zones may be longer than the height of a single DNA nucleotide in a single-stranded polynucleotide, and therefore a current signal that a nanopore can generate may be dependent on more than one nucleotide, for example 2, 3, 4, 5, 6, 7 or more nucleotides. These nucleotides form what may be termed a “K-mer.” The number of possible K-mers for a DNA formed of 4 types of bases (e.g., A, T, C and G) is 4^{K}. In some cases, a DNA may be formed of more than 4 types of bases. For example, if base C at some positions may be methylated, the DNA is considered to be formed of 5 types of bases and the number of possible K-mers is 5^{K}.

The recognition zone of a nanopore may be generally considered as sensing a certain number of bases in a polynucleotide that span the constriction of the nanopore. For example, a MspA nanopore is generally considered as primarily sensing at least a four-nucleotide sequence when ssDNA alone is translocating relative the nanopore. However, if a duplex end is positioned adjacent to the nanopore constriction, then both duplex nucleotides in addition to ssDNA nucleotides traversing the constriction can regulate the ionic current because the bulkiness of the duplex end will affect ionic current. In such instances, the current flowing through the pore would then be regulated by both dsDNA and ssDNA, such that the ssDNA, as well as the duplex dsDNA containing the incorporated nucleotide as a whole determine the current.

In some embodiments, the ionic current created by each K-mer may correspond with the particular sequence of the K-mer. In some cases, the ionic current is further influenced by a complementary oligonucleotide hybridized on the K-mer (for example, a complementary strand may be synthesized during sequencing operations according to some embodiments of the disclosed technology). Thus, one method of decoding a polynucleotide sequence includes obtaining a “K-mer map” and using the K-mer map to infer the sequences of unknown polynucleotides. Obtaining the “K-mer map” may include sampling each possible K-mer instance residing in the nanopore recognition zone, measuring the associated ionic current the K-mer creates, and constructing/extracting a unique code for the K-mer based on the measured current. In some examples, a look-up table may be used to summarize the codes that correspond to all the possible K-mers. In some examples, decoding a polynucleotide sequence may be facilitated by machine learning processes (e.g., using a supervised learning model).

In some embodiments, a predetermined K-mer map may be stored in the sequencer controller 1011, the computer 102, or the network/cloud 103 and may be used to aid the sequence identification of previously unmeasured polynucleotides. In some embodiments, a K-mer map may be determined/generated as a polynucleotide with a known sequence, e.g., a de Bruijn sequence designed for sampling all possible instances of K-mer, is measured in the example nanopore sequencing system. In some embodiments, a nanopore sequencing system can use machine learning/deep learning methods to decode polynucleotide sequences (i.e., infer polynucleotide sequences based on the series of codes extracted from measured nanopore signals corresponding to the target polynucleotides). In one embodiment, a machine learning/deep learning is based upon a read information (the nanopore signal) corresponding to a particular single-stranded polynucleotide region plus a double-stranded polynucleotide region in the target polynucleotide. In the case of a DNA polymerase, the double-stranded polynucleotide is a dsDNA duplex. In the case of a RNA, the double-stranded polynucleotide is a DNA-RNA duplex. In another embodiment, the machine learning/deep learning is based upon the read information of a particular ss-DNA plus ds-DNA region in the target polynucleotide in combination with the K-mer map.

The controller 1011 can be implemented in software and/or in hardware. In some examples, the control functionalities of controller 1011, such as actuating a nanopore unit cell, signal detection from a nanopore unit cell, or accessing or controlling nanopore sensors in an array, can be implemented by electronic hardware, computer software, or combinations of both. Whether such functionalities are implemented by hardware or software depends upon the particular application and design constraints imposed on the overall system. The described functionalities can be implemented in varying ways for each particular application, but such implementation decisions should not be interpreted as causing a departure from the scope of the disclosure.

In some examples, the control functionalities of controller 1011 can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor or group of processors for performing the methods described herein may be of various types including programmable devices (e.g., CPLDs and FPGAs) and non-programmable devices such as gate array ASICs or general-purpose microprocessors.

A processor can also be implemented as a combination of computing devices, e.g., a combination of a DSP and a microprocessor, a plurality of microprocessors, one or more microprocessors in conjunction with a DSP core, or any other such configuration. For example, systems described herein may be implemented using a discrete memory chip, a portion of memory in a microprocessor, flash, EPROM, or other types of memory. In some examples, a hardware platform for providing a computational environment may be used. The hardware platform may comprise a processor (e.g., CPU) and a memory such as random access memory (RAM). In some embodiments, graphics processing units (GPUs) can be used. In some embodiments, hardware platforms for performing computational methods as described herein comprise one or more computer systems with one or more processors. In some embodiments, smaller computers are clustered together to yield a supercomputer network. The hardware platform may be specially constructed for the required purposes, or it may be a general-purpose computer (or a group of computers) selectively activated or reconfigured by a computer program and/or data structure stored in the computer. In some embodiments, a group of processors performs some or all of the described functionalities collaboratively (e.g., via a network or cloud computing) and/or in parallel.

Elements of the methods or processes described herein can be embodied in a software module executed by a processor. A software module can reside in RAM memory, flash memory, ROM memory, EPROM memory, EEPROM memory, registers, hard disk, a removable disk, a CD-ROM, or any other form of computer-readable storage medium. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions.

FIG. 1B illustrates a portion of one of the nanopore unit cells. The nanopore unit cell may include a membrane 1124 formed from any suitable natural and/or synthetic material. The membrane 1124 may be formed of a non-permeable or semi-permeable material. In an example, the membrane 1124 includes a bilipid layer or a block copolymer structure. The nanopore unit cell may further include a nanopore 1123, which may be any of the biological nanopores, solid-state nanopores, hybrid nanopores, and synthetic nanopores described herein. In an example, the nanopore 1123 may be a hollow defined by, for example: a polynucleotide structure, a polypeptide structure, or a solid-state structure, e.g., a carbon nanotube, which is disposed in the membrane 1124. In a further example, the membrane 1124 is a synthetic membrane (e.g., a solid-state membrane, one example of which is silicon nitride), and the nanopore 1123 is in a hollow extending through the membrane 1124. In one embodiment, a protein MspA may be inserted into a pre-formed membrane (e.g., formed of block copolymers).

The membrane 1124 separates the nanopore unit cell into a cis compartment/well and a trans compartment/well. A target polynucleotide can translocate from the cis well, relative the nanopore 1123, to the trans well. A cis electrode 1130 is associated with the cis compartment; and a trans electrode 1134 is associated with the trans compartment. The electrodes may be used to apply a voltage across the nanopore, thus driving ionic current flows through the nanopore 1123 and exerting an electric force on the target polynucleotide. In some examples, the electrodes are faradaic electrodes. In some examples, the electrodes are non-faradaic electrodes. A current detector 1122 may be used to measure the ionic current through the nanopore and the detected signal may be transmitted to the sequencer controller 1011.

The cis electrode 1130 that is used depends, at least in part, upon the redox couple in the electrolyte. As examples, the cis electrode may be gold (Au), platinum (Pt), carbon (C) (e.g., graphite, diamond, etc.), palladium (Pd), silver (Ag), copper (Cu), or the like. In an example, the cis electrode 1130 may be a silver/silver chloride (Ag/AgCl) electrode. In one example, one cis compartment is associated with one nanopore. In some examples, one cis compartment may be shared by an array of nanopore unit cells. The trans electrode 1134 that is used depends, at least in part, upon the redox couple in the electrolyte. As examples, the trans electrode may be gold (Au), platinum (Pt), carbon (C) (e.g., graphite, diamond, etc.), palladium (Pd), silver (Ag), copper (Cu), or the like. In an example, the trans electrode 1134 may be a silver/silver chloride (Ag/AgCl) electrode. In one example, one trans compartment is associated with one nanopore. In some examples, one trans compartment may be shared by an array of nanopore unit cells.

In some examples, the relevant electrochemical half-reactions at the electrodes for a Ag/AgCl electrode in NaCl or KCl solution, are:

Cis(cathode): AgCl+e⁻→Ag⁰+Cl⁻; and

Trans(anode):Ag⁰+Cl⁻→AgCl+e⁻.

For every unit charge of current, one Cl atom is consumed at the trans electrode. Though the discussion above is in terms of an Ag/AgCl electrode in NaCl or KCl solution, it is to be understood that any electrode/electrolyte pair that may be used to pass the current may apply.

In use, an electrolyte may be filled into the cis well and the trans well. In alternative examples, the electrolyte in the cis well and the trans well may be different. The electrolyte may be any electrolyte that is capable of dissociating into counter ions (a cation and its associated anion). As examples, the electrolyte may be an electrolyte that is capable of dissociating into a potassium cation (K⁺) or a sodium cation (Na⁺). This type of electrolyte includes a potassium cation and an associated anion, or a sodium cation and an associated anion, or combinations thereof. Examples of potassium-containing electrolytes include potassium chloride (KCl), potassium ferricyanide (K₃[Fe(CN)₆]·3H₂O or K₄[Fe(CN)₆]·3H₂O), or other potassium-containing electrolytes (e.g., bicarbonate (KHCO₃) or phosphates (e.g., KH₂PO₄, K₂HPO₄, K₃PO₄). Examples of sodium-containing electrolytes include sodium chloride (NaCl) or other sodium-containing electrolytes, such as sodium bicarbonate (NaHCO₃), sodium phosphates (e.g., NaH₂PO₄, Na₂HPO₄ or Na₃PO₄). As another example, the electrolyte may be any electrolyte that is capable of dissociating into a ruthenium-containing cation (e.g., ruthenium hexamine, such as [Ru(NH₃)₆]²⁺ or [Ru(NH₃)₆]³⁺) Electrolytes that are capable of dissociating into a lithium cation (Li⁺), a rubidium cation (Rb⁺), a magnesium cation (Mg⁺), or a calcium cation (Ca⁺) may also be used.

Accordingly, disclosed systems for identifying a base sequence of a template polynucleotide may include an instrument comprising a nanopore in contact with a solution (e.g., residing in a membrane and partially immersed in an electrolyte), and a processor configured to selectively move the template polynucleotide relative to nanopore and configured to measure a pore current (i.e., ionic current passing through the nanopore) of a single-strand portion of the template polynucleotide and a duplex portion of the template polynucleotide and the complementary polynucleotide; and a non-volatile data storage medium coupled to the instrument, wherein the non-volatile data storage medium stores a K-mer map, the K-mer map comprising a plurality of entries in which each entry includes a K-mer sequence and a pore current at an applied read potential, the K-mer sequence includes at least one position (e.g., nucleobase or nucleotide) of the template polynucleotide in the single-strand portion and at least one position (e.g., nucleobase or nucleotide) of the template polynucleotide in the duplex portion. In some embodiments, instrument comprises the non-volatile data storage medium. In some embodiments, the processor is further configured to determine the base sequence of the template polynucleotide based on the measured pore current and the K-mer map. In some embodiments, disclosed systems may further include a computer attached locally to the instrument, wherein the computer comprises the non-volatile data storage medium. In some embodiments, disclosed systems may further include a computer networked to the instrument, wherein the computer comprises the non-volatile data storage medium. In some embodiments, disclosed systems may further include a computer attached locally to the instrument, wherein the computer is further configured to determine the base sequence of the template polynucleotide based on the measured pore current and the K-mer map.

### Sequencing Processes

In some sequencing processes of the disclosed technology, determining the polynucleotide sequence may be based on the pore current level (e.g., means value) measured under a single read potential or under multiple read potentials. In alternative sequencing processes of the disclosed technology, determining the polynucleotide sequence may be based on variations/noise of the pore current determined under a single read potential or under multiple read potentials. It is to be understood that any combination of the above may be utilized in certain sequencing processes of the disclosed technology.

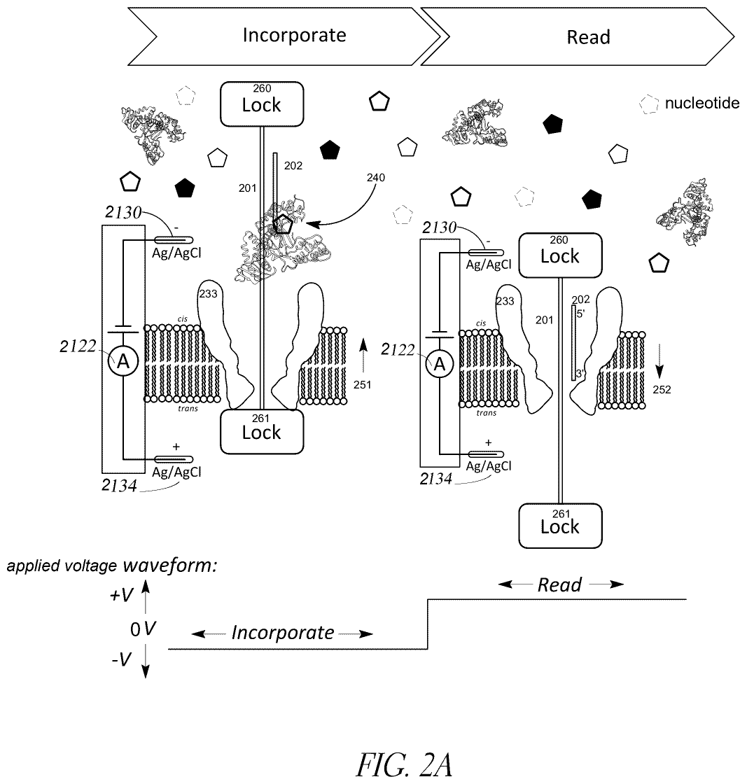

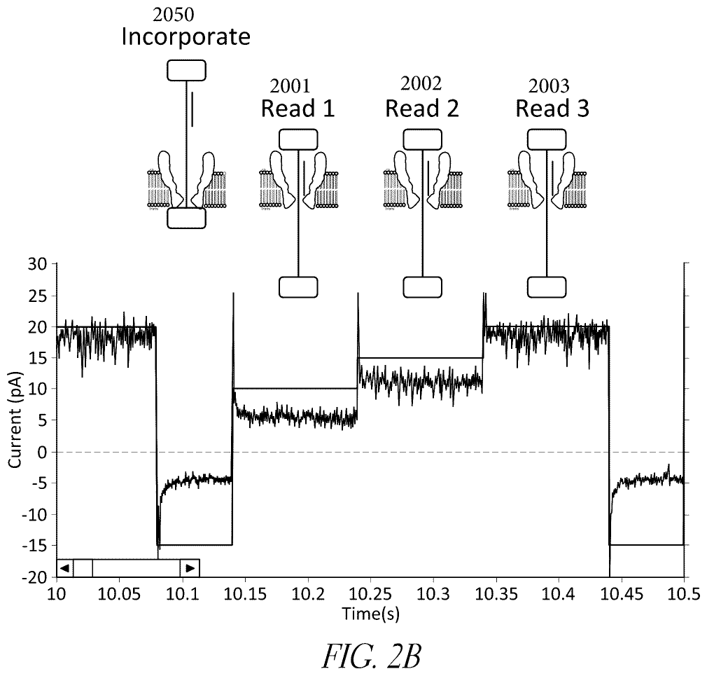

FIG. 2A illustrates example sequencing operations used in some embodiments of the disclosed technology. As shown, a single-stranded target polynucleotide 201 (such as DNA or RNA) to be sequenced is disposed through the aperture of a nanopore 233. A complementary strand 202 may be synthesized on a portion of the target polynucleotide to form a duplex, in the cis side of the nanopore 233. One or more structural “lock” moieties 260 and 261 may be attached to the target polynucleotide 201. The structural “lock” moieties may include a streptavidin or a hairpin loop, for example. The one or more structural “lock” moieties 260 and 261 may help keep the complex of the single-stranded target polynucleotide 201-complementary strand 202 close to the nanopore while the complex is subject to electric forces and moved upwards or downwards relative to the nanopore. In certain embodiments, one or more structural “lock” moieties is utilized to isolate a region of a template strand for sequencing through a nanopore. A cis electrode 2130 is associated with the cis side; and a trans electrode 2134 is associated with the trans side. The electrodes may be used to apply a voltage across the nanopore, thus driving ionic current flows through the nanopore 233 and exerting an electric force on the target polynucleotide. In some examples, the electrodes are faradaic electrodes. In some examples, the electrodes are non-faradaic electrodes. A current detector 2122 may be used to measure the ionic current through the nanopore.

As shown in FIG. 2A, in a position permitting incorporation, where the 3′ end of the complementary strand 202 is placed in a location that is accessible to the polymerase and free nucleotides in the electrolyte solution, a polymerase 240 in the electrolyte solution can bind to the target polynucleotide and the complementary strand 202 may be extended. The rate of complementary strand extension may depend on the concentrations of polymerase and nucleotides in the electrolyte solution, compositions of the electrolyte solution, temperature, competitive binding moieties, etc. In some embodiments, the disclosed technology utilizes a low rate of strand extension (e.g., by using low concentrations of polymerase and nucleotides), such that within a predetermined amount of time (before the target polynucleotide is moved downwards to lodge at the nanopore), it is highly probable that only one nucleotide, or no nucleotide, would have been successfully incorporated. Thus, the disclosed technology can minimize the occurrence of two or more nucleotides being incorporated during the duration of target-complementary complex in the “incorporate” position.

After a predetermined amount of time, the sequencing process is moved to a “read” position, where a downward electric force 252 is applied on the target polynucleotide that lodges the duplex within the aperture of the nanopore 233, and an electrical measurement is made. In the read position, the polymerase 240 is dislodged from the target polynucleotide so that the complementary strand 202 cannot be extended with a nucleotide during the read position. A measurement is then made, for example, the ionic current through the nanopore. The particular measurement value of that measurement is based on the particular complementary bases that are located near the 3′ end of the complementary strand 202 plus the particular sequence of one or more bases that are in a single-stranded portion of the target polynucleotide 201 as a whole, within or near the aperture of the nanopore. Accordingly, the particular measurement value provides information from which the sequence of the bases in the target polynucleotide may be determined. In certain embodiments, the read position determines whether or not a particular base has been incorporated during the incorporate position.

After the measurement is made, an upward electric force 251 may be applied on the target polynucleotide that dislodges the duplex from the aperture of the nanopore so that in another incorporate position, a polymerase 240 from the electrolyte solution can bind to the target polynucleotide, and the complementary strand 202 may be extended again. Then, the measurement may be repeated in another read position.

The repeated measurements provide further information from which the sequence of the bases in the target polynucleotide may be determined. The disclosed technology is compatible with relatively long reads, e.g., of up to about 1,000 bases, or up to about 2,000 bases, or up to about 5,000 bases, or even up to 10,000 bases or more. The disclosed technology may enable controllable duration of the incorporation positions and duration of the read positions so that a read position measures incorporation of a single nucleotide. For example, the duration of an incorporation position may be set to correspond to a time for a single incorporation of a nucleotide. For example, the duration of an incorporation position may be set to reduce multiple incorporation of nucleotides prior to being read. Thus, non-limiting advantages of the disclosed technology include: allowing for controllable measurements; improving the temporal precisions of the measurements; and avoiding translocation events that are too fast to be detected (which may lead to detection errors or other types of errors that detrimentally affect sequencing accuracy). In some embodiments, the disclosed technology may be used to identify modified bases, such as methylated bases, without the need to chemically or enzymatically modify the modified bases.

FIG. 2B shows an experimental result measured according to the example sequencing operations illustrated in FIG. 2A. The ionic current through the nanopore as a function of time is recorded for one incorporate position (2050) and three different read positions. The three different read positions, “read 1” (2001), “read 2” (2002) and “read 3” (2003), are measured under three different applied voltage values. The applied voltage values can be selected to be any suitable applied voltage. In the incorporate position, only a single strand of a template polynucleotide is threaded through the pore. Without being bound by theory, the current measured in the incorporate position is influenced by the interaction between the “trans” lock moiety 261 and the “trans” portion of the pore protein, as illustrated in FIG. 2A. In a read position, a single-stranded portion of the template polynucleotide template and a duplex portion of the template polynucleotide and the complementary polynucleotide determine the pore current.

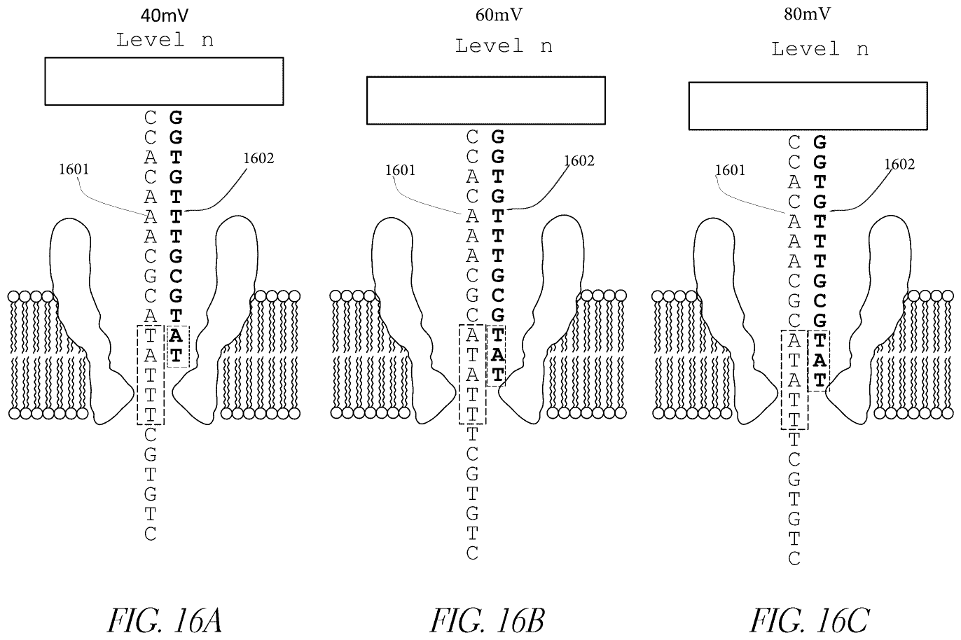

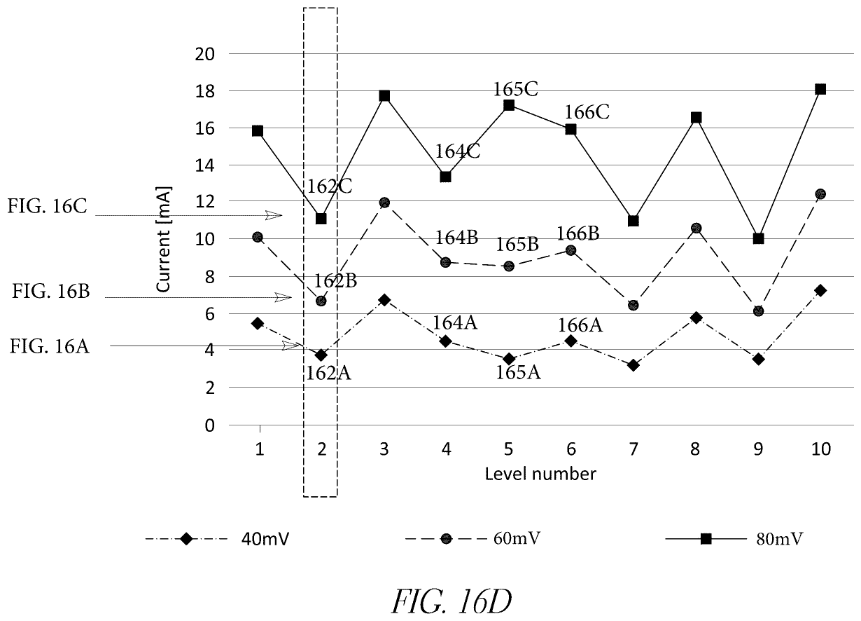

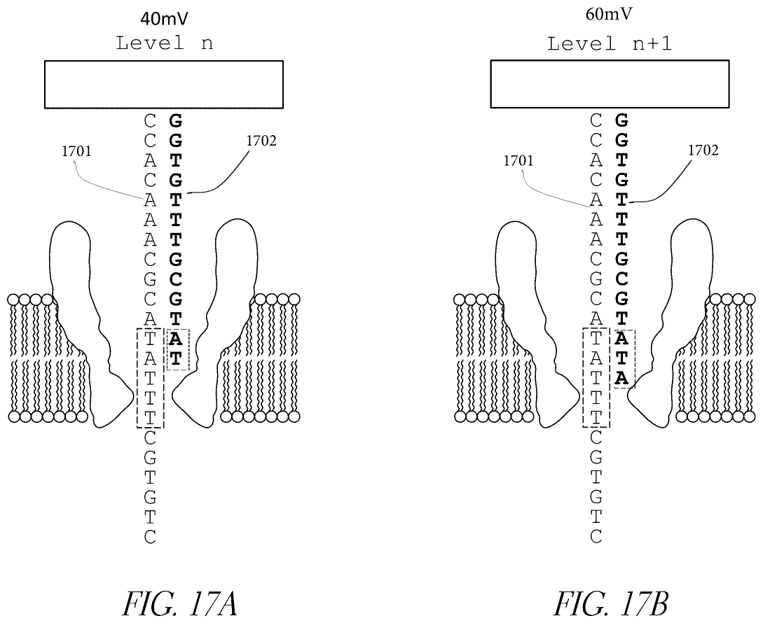

In some cases, the three ionic current traces recorded under the three different applied voltages may exhibit non-Ohmic behavior. That is, the three ionic current traces may not simply scale with the value of the applied voltages. The non-Ohmic behavior may be partly due to voltage-dependent modulation of the relative position and/or interactions between the polynucleotide and the nanopore. As a result, nonredundant, uncorrelated or independent pieces of information for the polynucleotide sequence may be obtained through measurements using different applied voltages. Without being bound by theory, FIG. 16A, FIG. 16B and FIG. 16C illustrate examples of how the applied voltage can modulate the relative position between the polynucleotide and the nanopore and what portions of the polynucleotide interact with the nanopore “read head”. After a certain number of nucleotide incorporation cycles, base “T” has been incorporated into the complementary strand show in FIG. 16A, FIG. 16B and FIG. 16C. The applied voltage is 40 mV, 60 mV and 80 mV for FIG. 16A, FIG. 16B and FIG. 16C, respectively.

In FIG. 16A, the portion “TATTT” in the template strand 1601 and the portion “AT” in the complementary strand 1602 (indicated by the dashed boxes) are the regions of the polynucleotide that affect the measured ionic current trace. The mean ionic current corresponding to the situation of FIG. 16A (e.g., averaged over the whole time period of “read 1” illustrated in FIG. 2B) is indicated by reference numeral 162A in FIG. 16D.

In FIG. 16B, the portion “ATATT” in the template strand 1601 and the portion “TAT” in the complementary strand 1602 (indicated by the dashed boxes) are the regions of the polynucleotide that affect the measured ionic current trace, since the larger applied voltage pulls the polynucleotide further down relative to the nanopore as compared to the situation shown in FIG. 16A. A shorter ssDNA region (“TT” in FIG. 16B, as compared to “TTT” in FIG. 16A) affects the nanopore current as the applied voltage increases. The mean ionic current corresponding to the situation of FIG. 16B (e.g., averaged over the whole time period of “read 2” illustrated in FIG. 2B) is indicated by reference numeral 162B in FIG. 16D.

In FIG. 16C, an even larger voltage is applied, and while the portion “ATATT” in the template strand 1601 and the portion “TAT” in the complementary strand 1602 (indicated by the dashed boxes) are still the regions of the polynucleotide that affect the measured ionic current trace, they are pulled further down relative to the nanopore as compared to the situation shown in FIG. 16B. Thus, the polynucleotide can interact with the nanopore differently between the situation of FIG. 16B and the situation of FIG. 16C. The mean ionic current corresponding to the situation of FIG. 16C (e.g., averaged over the whole time period of “read 3” illustrated in FIG. 2B) is indicated by reference numeral 162C in FIG. 16D.

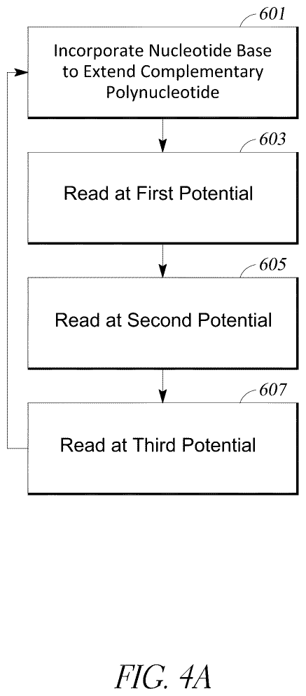

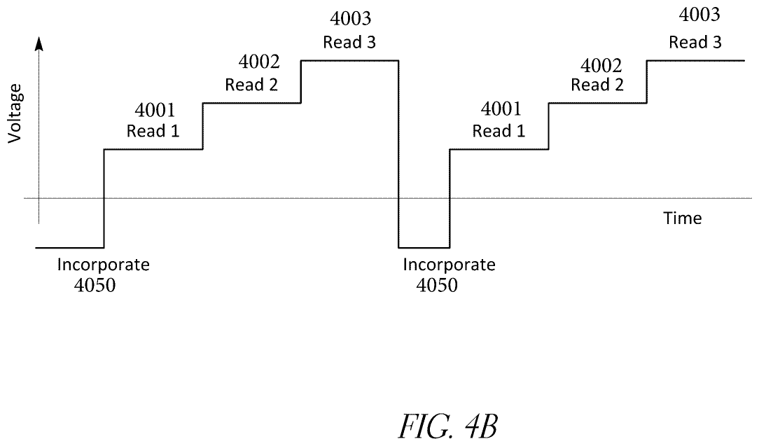

FIG. 16D shows the mean nanopore ionic current as a function of “level number” for the polynucleotide illustrated in FIG. 16A, FIG. 16B and FIG. 16C under a 40 mV, 60 mV or 80 mV applied voltage. The level number represents the number of nucleotides that has been incorporated into the complementary strand after a series of “incorporation” operations shown in FIG. 2A, FIG. 2B, FIG. 4B or FIG. 7A. A non-Ohmic behavior of the ionic current as the applied voltage increases from 40 mV to 60 mV and 80 mV is observed in FIG. 16D. At least for some of the level numbers, the mean ionic current does not scale linearly with the magnitude of the applied voltage. Moreover, the applied voltage affects the interaction between the polynucleotide and the nanopore in an unexpected way, as exemplified by how point 165C is higher than both points 164C and 166C under the 80 mV applied voltage, while point 165B is lower than both points 164B and 166B under the 60 mV applied voltage and point 165A is lower than both points 164A and 166A under the 40 mV applied voltage.

FIG. 16D also illustrates how using the collective information obtained from measurements under more than one applied voltage is particularly useful in determining the polynucleotide sequence. For example, point 164A and point 166A have very similar values, so it would be difficult to distinguish between the situation of level number 4 and the situation of level number 6 and identify the polynucleotide sequence based on the mean ionic current measured under the 40 mV applied voltage alone. However, with the additional information provided by points 164B, 164C, 166B and 166C, it is possible to distinguish between the situation of level number 4 and the situation of level number 6 and identify the polynucleotide sequence.

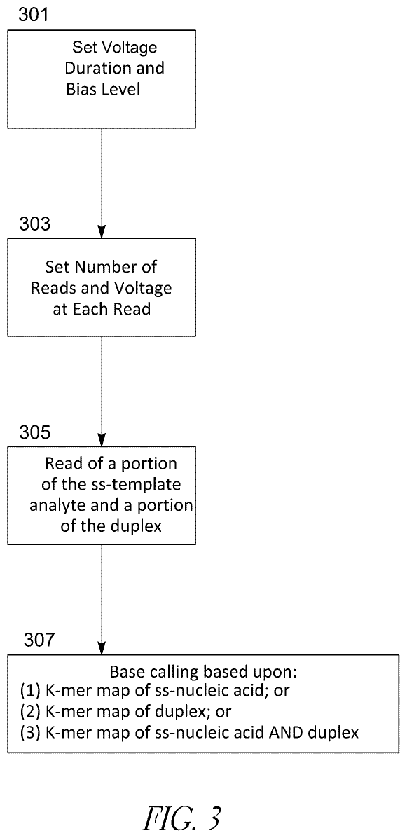

FIG. 3 illustrates a method for tailored sequencing according to some embodiments of the disclosed technology, which may involve adjusting the incorporate and/or read position duration, the applied bias voltages, or the K-mer map complexity. The method may start at block 301, where the applied voltage duration and bias level are set. Since there is no ratchet/motor protein to control translocation of, for example, a single stranded template through the nanopore, the time period of the incorporation and read positions can be adjusted. The waveform applied to the cis/trans electrodes may generally be DC (with one polarity for an incorporate position and with another polarity for a read position), such as in a stepped waveform. There may be slight AC waveform on top of the DC waveform to induce slight movement of the duplex within the nanopore. This slight AC waveform on top of the DC waveform may increase the noise, but also increase the information in the read.

In some embodiments, the disclosed method is directed to a DNA template in which a complementary DNA strand is extended by a DNA polymerase. In certain embodiments, a pore current is read when a single-stranded portion of the DNA template and a duplex portion of the DNA template and the complementary strand are held in a (non-translocating) position near the nanopore, e.g., within the nanopore such that extension of the complementary strand cannot occur. In some embodiments, the disclosed method is directed to with a RNA template in which a complementary DNA strand is extended by a reverse transcriptase. In certain embodiments, a pore current is read when a single-stranded portion of the RNA template and a duplex portion of the RNA template and the complementary DNA strand are held in a (non-translocating) position near the nanopore, e.g., within the nanopore such that extension of the complementary DNA strand cannot occur.

After setting the applied voltage duration and bias level, the method may then move to block 303, where the number of reads and voltage at each read are set. The number of reads and the voltage at each read, the time for incorporation, and the time for each read can all be adjusted to determine the quality of sequencing and the speed of sequencing, for example. The method may then move to block 305, reading of a portion of the single stranded template analyte and a portion of the duplex. The method may then move to block 307, base calling based upon: (1) a K-mer map of a single-stranded portion of the template polynucleotide near the pore; or (2) a K-mer map of a portion of the duplex of the template polynucleotide and the complementary polynucleotide; or (3) a K-mer map of the single-stranded portion and the duplex portion near the pore.

FIG. 4A illustrates another method for sequencing a polynucleotide according to some embodiments of scanning the template polynucleotide at more than one read potential consistent with the experiment associated with FIG. 2B, which involves reading a portion of the polynucleotide at three different applied voltages. The method may include block 601, incorporating an additional nucleotide to the complementary polynucleotide, which includes a single-stranded portion of the template polynucleotide and a duplex portion the template polynucleotide and the complementary polynucleotide (such as shown in FIG. 2A). The method may then move to block 603, reading the pore current at a first electric potential difference. The method may then move to block 605, reading the pore current at a second electric potential difference. The method may then move to block 607, reading the pore current at a third electric potential difference. The method may then move back to block 601 and start another cycle of sequencing. Although FIG. 4A shows reading the pore current three different times at three different applied read potential, the number of read potentials after each incorporation potential can be a single read potential or can be any number of multiple read potentials. For example, a single read potential after each incorporation potential can be used to increase the throughput of sequencing operations. For example, multiple read potentials after each incorporation potential can be used to increase the accuracy of sequencing operations. It is understood that the during the incorporation potential or incorporation position, the complementary strand may or may not be extended by incorporation of a free nucleotide. If a free nucleotide is incorporated during the incorporation potential or incorporation position, the pore current during read position can detect a different read signal at one or more read potentials.

FIG. 4B illustrates certain embodiments of the applied voltage as a function of time that may be used in connection with the method shown with FIG. 4A. As shown, the applied voltage may start with a negative incorporation potential to move the target-complementary complex to the incorporate position (4050), and then proceed to read the nanopore signal at three different positive applied voltages, “read 1” (4001), “read 2” (4002) and “read 3” (4003), while the target-complementary complex is lodged at the nanopore by the application of the positive voltages. The pattern of applied voltage may then repeat.

Accordingly, disclosed methods of sequencing a template polynucleotide using a nanopore may include incorporating a nucleotide to extend a complementary polynucleotide; reading a first pore current at a first applied read potential when a single-strand portion of the template polynucleotide and a duplex portion of the template polynucleotide and a complementary polynucleotide are held in a (non-translocating) position near the nanopore, e.g., within the nanopore such that extension of the complementary polynucleotide cannot occur; reading a second pore current at a second applied read potential when the single-strand portion of the template polynucleotide and the duplex portion of the template polynucleotide and the complementary polynucleotide are held in a (non-translocating) position near the nanopore, e.g., within the nanopore such that extension of the complementary polynucleotide cannot occur; comparing the first pore current and the second pore current to a K-mer map, the K-mer map comprising a plurality of entries in which each entry includes a K-mer sequence and a plurality of pore currents at a plurality of applied read potential, the K-mer sequence includes at least one position of the template polynucleotide in the single-strand portion and at least one position of the template polynucleotide in the duplex portion; and determining a sequence of the template polynucleotide based upon the comparison of the pore current to the K-mer map. In some embodiments, the K-mer sequence of the K-mer map comprises at least two positions of the template polynucleotide in the single-strand portion. In some embodiments, the disclosed methods may further include determining a variation (e.g., a coefficient of variation) of the read pore current, wherein the sequence of the template polynucleotide is further determined based upon the variation (e.g., the coefficient of variation). In some alternative embodiments, the disclosed methods may determine a variation (e.g., a coefficient of variation) of the pore current and determine the sequence of the template polynucleotide based upon the variation (e.g., the coefficient of variation) alone. In some embodiments, the disclosed methods may further include identifying a modified base, such as a methylated-C base, in the determined sequence by an incorporation time of a nucleotide to extend the complementary polynucleotide. In some embodiments, the nucleotide is incorporated to extend the complementary polynucleotide when a 3′ end of the complementary polynucleotide is not sequestered within (e.g., positioned away from) the nanopore such that the 3′ end is placed in a location that is accessible to the polymerase and free nucleotides in the electrolyte solution for a set period of time. In some embodiments, the pore current is read when a 3′ end of the complementary polynucleotide is biased away from the nanopore (i.e., separated from the nanopore via the application of a voltage bias) for a set period of time.

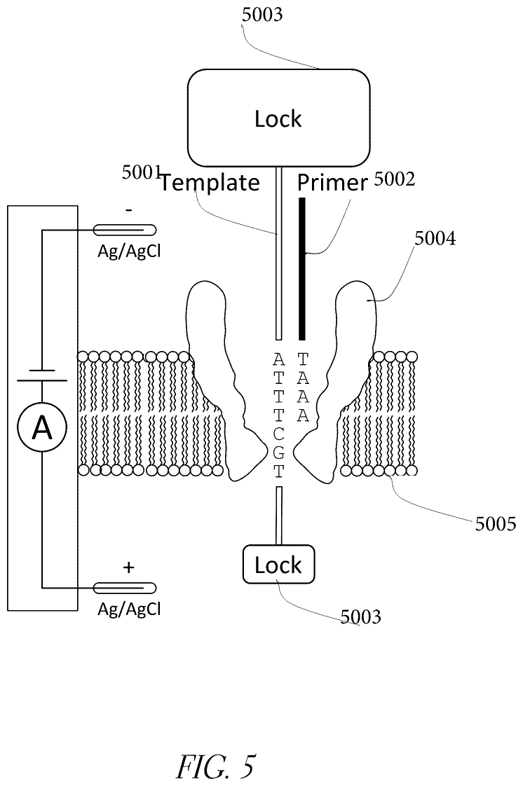

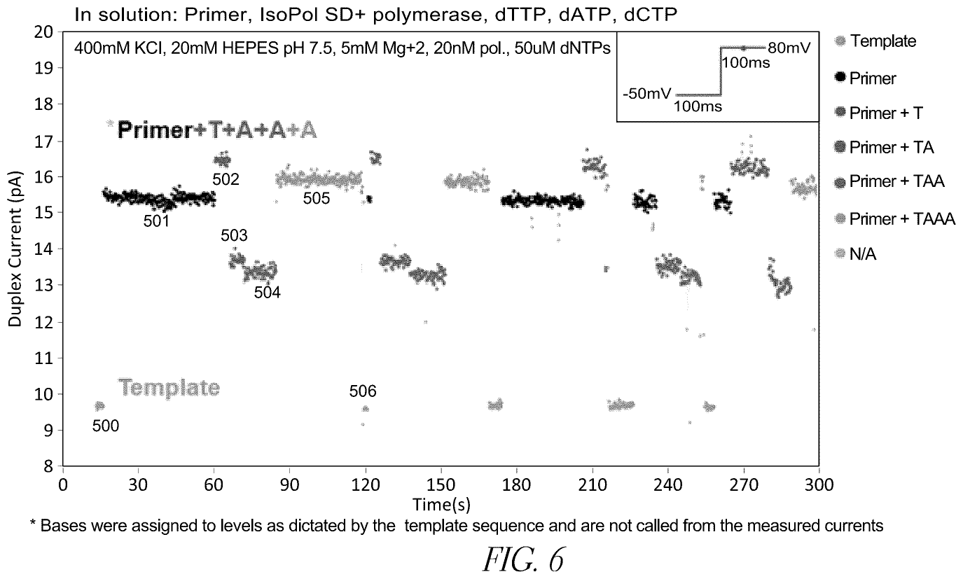

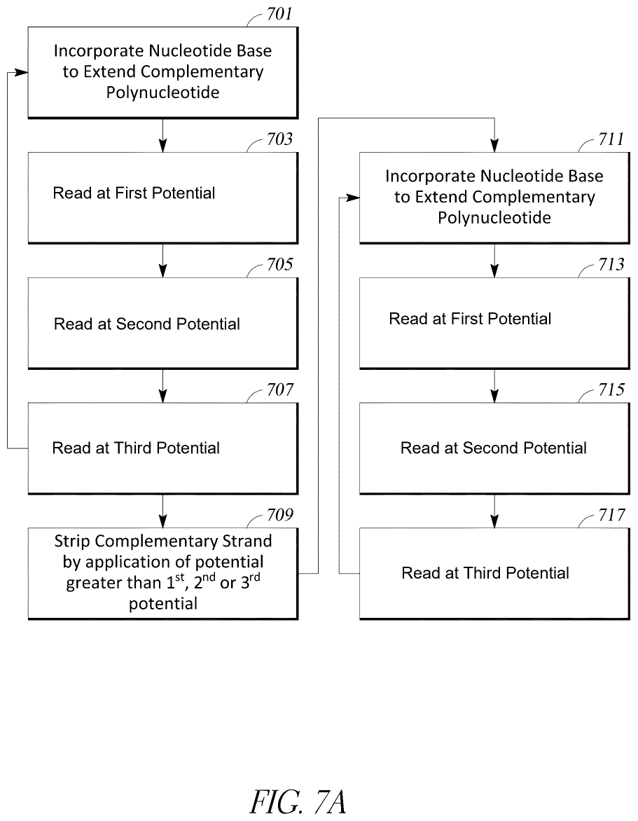

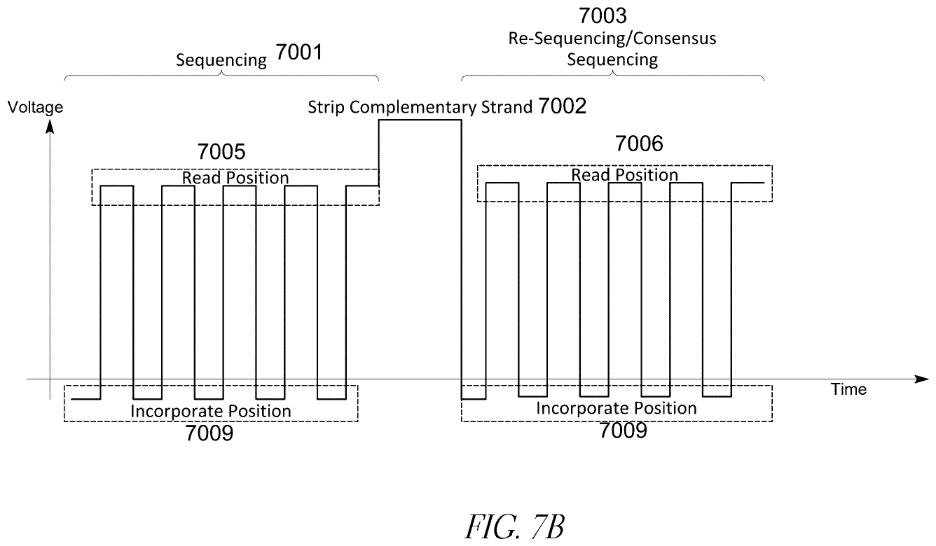

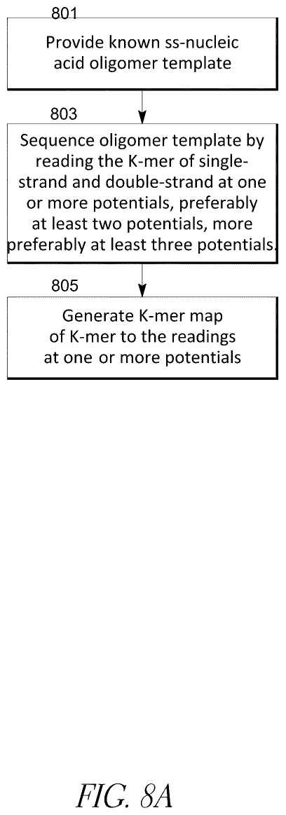

FIG. 5, FIG. 6, FIG. 7A and FIG. 7B illustrate some embodiments involving re-sequencing or consensus sequencing of a polynucleotide in order to sequence the template polynucleotide more than once.

As shown in FIG. 5, a single-stranded template polynucleotide including the sequence “ATTTCGT” is disposed through the aperture of a nanopore. The single-stranded template polynucleotide (5001) may include a primer binding site which was added during a nucleic acid library preparation process. A complementary strand including the sequence “TAAA” is extended from a primer (5002) hybridized to the primer binding site on the template. The complementary strand is base paired with a portion of the template polynucleotide to form a duplex. Two structural “lock” moieties (5003) are attached to the two ends of the template polynucleotide. In this particular example, the portion “ATTTCGT” in the template polynucleotide and the portion “TAAA” in the complementary strand collectively influence the measured ionic current through the nanopore formed in a pore protein (5004) deposited in a membrane (5005).

In some embodiments, the single-stranded template polynucleotide may further include a calibration sequence and/or barcode sequence which was added during the nucleic acid library preparation process. The calibration sequence and/or barcode sequence may be a known sequence including a sufficient number of diverse nucleobase combinations. Thus, measurements on the calibration sequence may be used to calibrate or normalize the signal obtained in each individual nanopore unit cell. The barcode sequence can be used to identify the template polynucleotide when multiplexing a plurality of single-stranded template polynucleotides through one nanopore unit cells or through an array of nanopore unit cells.

FIG. 6 shows experimental results measured according to the example illustrated in FIG. 5. The experiment producing FIG. 6 uses sequencing operations (as explained with FIG. 2A) where a duration of an incorporate position is 100 ms under an applied voltage of −50 mV; and a duration of a read position is 100 ms under an applied voltage of 80 mV. Each data point (dot) in FIG. 6 represents the value of measured ionic current averaged over 100 ms. In certain embodiments, any suitable durations and any suitable applied voltages of the incorporate position and the read position may be used. The dots in group 500 correspond to a state where only the template is in the nanopore (no duplex is formed). The dots in group 501 correspond to a state where the template and the primer duplex is measured in the nanopore. The dots in group 502 correspond to a state where the template and the primer+T is measured in the nanopore. The dots in group 503 correspond to a state where the template and the primer+TA duplex is measured in the nanopore. The dots in group 504 correspond to a state where the template and the primer+TAA duplex is measured in the nanopore. The dots in group 505 correspond to a state where the template and the primer+TAAA duplex is measured in the nanopore. That the state of the polynucleotide has changed is indicated by (or inferred from) the discrete changes in the nanopore signal (e.g., the average ionic current). The complementary strand may be stripped away from the template by using a downward electric force to pull the duplex against the nanopore. Then, the template alone may be measured again (represented by group 506). Then, another primer may be hybridized to the template and another round of primer extension and sequencing may be performed. FIG. 6 also indicates the waiting time before an additional nucleotide is successfully incorporated. For example, the state represented by group 504 lasts for about 15 seconds before another base A is successfully incorporated and the state is moved to the one represented by group 505. FIG. 6 shows that the nucleic acid sequence of regions of repeated incorporation of nucleotides can be identified. FIG. 6 shows that the complementary strand may be stripped from the template and that a second series of incorporation positions and read positions may be performed for re-sequencing or consensus identification of the template polynucleotide.

FIG. 7A illustrate a method of sequencing which involves re-sequencing or consensus sequencing of a polynucleotide. The method may include block 701, incorporating an additional nucleotide to the duplex, which includes a portion of the single stranded template analyte and a portion of the complementary strand (such as shown in FIG. 5). The method may then move to block 703, reading the duplex at a first electric potential difference. The method may then move to block 705, reading the duplex at a second electric potential difference. The method may then move to block 707, reading the duplex at a third electric potential difference. The method may then move back to block 701 and start another cycle of sequencing. After a certain number of sequencing cycles, the method may move to block 709, stripping/removing the complementary strand away from the single stranded template analyte by pulling the polynucleotide against the nanopore (e.g., by application of an electric potential difference greater than the first, second or third electric potential differences). Thus, the single stranded template analyte may be sequenced again during another round of primer extension reactions. The method may include block 711, incorporating an additional nucleotide to the duplex. The method may then move to block 713, reading the duplex at a first electric potential difference. The method may then move to block 715, reading the duplex at a second electric potential difference. The method may then move to block 717, reading the duplex at a third electric potential difference. The method may then move back to block 711 and start another cycle of sequencing. FIG. 7A shows conducting a first cycle of one or more read positions after each incorporation position for identification of the template polynucleotide, stripping/removing of the complementary polynucleotide, and conducting a second cycle of one of one or more read position after each incorporation potential for re-sequencing or consensus sequencing of the template polynucleotide.

FIG. 7B illustrates certain embodiments of the potentials of sequencing (7001) and re-sequencing (7003) of a template polynucleotide. The read position (7005) of the first sequencing comprises one or more read potential levels. The read position (7006) of the re-sequencing or consensus sequencing comprises one or more read potential levels. For example, the plurality of read potential levels as described in reference to FIG. 4B can be used. The read potential level(s) of the first sequencing versus re-sequencing/consensus sequencing may be the same or different.

Accordingly, disclosed methods of sequencing a template polynucleotide using a nanopore may include performing a plurality of cycles, each cycle comprising incorporating (7009) a nucleotide to extend the complementary polynucleotide to the template polynucleotide, and reading a pore current of a single-strand portion of the template polynucleotide and a duplex portion of the template polynucleotide and the complementary polynucleotide held in a (non-translocating) position near the nanopore, e.g., within the nanopore such that extension of the complementary polynucleotide cannot occur; stripping/removing (7002) the complementary polynucleotide from the template polynucleotide after one set of the plurality of cycles; and determining a second sequence of the template polynucleotide based on a current trace from the pore current reads of the plurality of cycles. In some embodiments, determining the sequence comprises comparing the current trace to a K-mer map, the K-mer map comprising a plurality of entries in which each entry includes a K-mer sequence and a pre-measured pore current at an applied read potential, the K-mer sequence includes at least one position of the template polynucleotide in the single-strand portion and at least one position of the template polynucleotide in the duplex portion. In some embodiments, the pore current is measured under more than one potential or under multiple potentials. In some embodiments, the K-mer sequence of the K-mer map comprises at least two positions of the template polynucleotide in the single-strand portion. In some embodiments, the disclosed methods may further include determining a variation (e.g., coefficient of variation) of the read pore current, wherein the sequence of the template polynucleotide is further determined based upon the variation (e.g., coefficient of variation). In some alternative embodiments, the disclosed methods may determine a variation (e.g., a coefficient of variation) of the pore current and determine the sequence of the template polynucleotide based upon the variation (e.g., the coefficient of variation) alone. In some embodiments, the disclosed methods may further include identifying a methylated-C base in the determined sequency by an incorporation time of a nucleotide to extend the complementary polynucleotide. In some embodiments, the nucleotide is incorporated to extend the complementary polynucleotide when a 3′ end of the complementary polynucleotide is not sequestered within (e.g., positioned away from) the nanopore such that the 3′ end is placed in a location that is accessible to the polymerase and free nucleotides in the electrolyte solution for a set period of time. In some embodiments, the pore current is read when a 3′ end of the complementary polynucleotide is biased away from the nanopore (i.e., separated from the nanopore via the application of a voltage bias) for a set period of time.

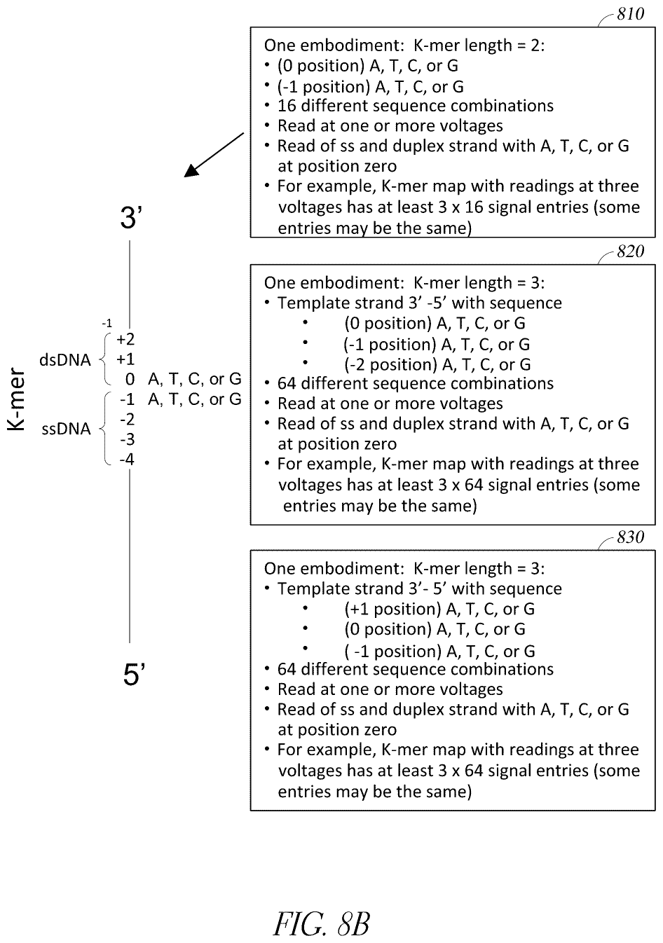

FIG. 8A illustrates a method of constructing a K-mer map for instances of K-mer including both a single stranded template strand region and a complementary strand region, and using the K-mer map for sequencing. The method may start at block 801, providing known single stranded nucleic acid oligomer template. The method may then move to block 803, sequencing the oligomer template by reading instances of K-mer at one or more electric potential differences, preferably at least two potential differences, more preferably at least three potential differences. The method may then move to block 805, generating K-mer map of K-mer to the readings at one or more potential differences. Blocks 801 to 805 may represent steps performed by a sequencing instrument maker. The constructed K-mer map can then be provided for base calling while sequencing unknown ss-nuclei oligomer analyte template. In some embodiments, the base calling may be performed by the sequencing instrument or by a computer coupled to the instrument with a K-mer map loaded or stored on the instrument or computer. In some embodiments, the base calling may be performed over the cloud (i.e., network) with a K-mer map loaded on the cloud server. FIG. 8B illustrates example K-mer instances and K-mer map constructions discussed in connection with FIG. 8A. In certain additional or alternative embodiments, the K-mer map used for decoding polynucleotide sequences may depend on the specific sequencing operation or experimental condition. In one embodiment, at one applied electric potential difference, the map is made of K-mers having a certain number of bases (such 5-mers); at another applied potential difference, the map is made up of K-mers having a different number of bases (such as 4-mers), since the applied electric potential difference may affect how the polynucleotide interact with the nanopore, such as the extent of the polynucleotide that is sensed by the nanopore recognition zone. In another embodiment, since the applied electric potential difference may shift the position of the polynucleotide relative to the nanopore recognition zone, the regions of the polynucleotide sensed by the recognition zone may be shifted/offset when different electric potential differences are applied, and thus different maps may be used when different electric potential differences are applied. For example, at one applied voltage, the regions of the polynucleotide sensed by the recognition zone may include a 5-mer in the template strand and 3 nucleotides in the complementary strand; at another applied voltage, the regions of the polynucleotide sensed by the recognition zone may include a 5-mer in the template strand and 2 nucleotides in the complementary strand.

In certain embodiments, the K-mer map 810 length is a 2-mer map of two nucleotide positions comprising a single base position in the single-stranded region (i.e., position −1) of the template stand and a single base position the double-stranded region (i.e., position 0) of the template strand. The position 0 indicates the nucleotide of the template strand in which a nucleotide has been incorporated into the complementary strand in an incorporation step prior to the current read step. The position −1 indicates the nucleotide of the template strand one base position from position 0 towards the 5′ end of the template strand. In certain embodiments, a 2-mer map of two nucleotide positions comprises 16 possible states of four different nucleotides (e.g., A, T, C, or G) or 4^{K} states. In certain embodiments, a 2-mer map with a single read step at a single read voltage following an incorporate step comprises 16 signal entries for decoding the incorporated nucleotide at position 0. In certain embodiments, a 2-mer map with a multiple M read steps at multiple read voltages following an incorporate step comprises M times 4^{K} signal entries for decoding the incorporated nucleotide at position 0. For example, a 2-mer map with three read steps following an incorporate step comprises 3 times 4^{K} entries or 48 signal entries for decoding the incorporated nucleotide at position 0. Although a K-mer map may have a same signal reading for two different bases at a single read voltage, the likelihood decreases for have a same set of signal readings for two different incorporated bases as the number of read steps of different read voltages increases.

In certain embodiments, the K-mer map 820 length is a 3-mer map of three nucleotide positions comprising two base positions in the single-stranded region (i.e., position −1 and position −2) of the template stand and a single base position of the double-stranded region (i.e., position 0) of the template strand. The position 0 indicates the nucleotide of the template strand in which a nucleotide has been incorporated into the complementary strand in an incorporation step prior to the current read step. The position −1 indicates the nucleotide of the template strand one base position from position 0 towards the 5′ end of the template strand. The position −2 indicates the nucleotide of the template strand two base positions from position 0 towards the 5′ end of the template strand.

In certain embodiments, the K-mer map 830 length is a 3-mer map of three nucleotide positions comprising a single base position in the single-stranded region (i.e., position −1) of the template stand and two base positions of the double-stranded region (i.e., position +1 and position 0) of the template strand. The position +1 indicates the nucleotide of the template strand in which a nucleotide has been incorporated into the complementary strand in two incorporation steps prior to the current read step. The position 0 indicates the nucleotide of the template strand in which a nucleotide has been incorporated into the complementary strand in an incorporation step prior to the current read step. The position −1 indicates the nucleotide of the template strand one base position from position 0 towards the 5′ end of the template strand.

In certain embodiments, a 3-mer map, such as K-mer map 820 or K-mer map 830, of three nucleotide positions comprises 64 possible states of four different nucleotides (e.g., A, T, C, or G) or 4^{K} states. In certain embodiments, a 3-mer map with a single read step at a single read voltage following an incorporate step comprises 64 signal entries for decoding the incorporated nucleotide at position 0. In certain embodiments, a 3-mer map with a multiple M read steps at multiple read voltages following an incorporate step comprises M times 4^{K} signal entries for decoding the incorporated nucleotide at position 0. For example, a 3-mer map with three read steps following an incorporate step comprises 3 times 4^{K} entries or 192 signal entries for decoding the incorporated nucleotide at position 0. Although a K-mer map may have a same signal reading for two different bases at a single read voltage, the likelihood decreases for have a same set of signal readings for two different incorporated bases as the number of read steps of different read voltages increases.

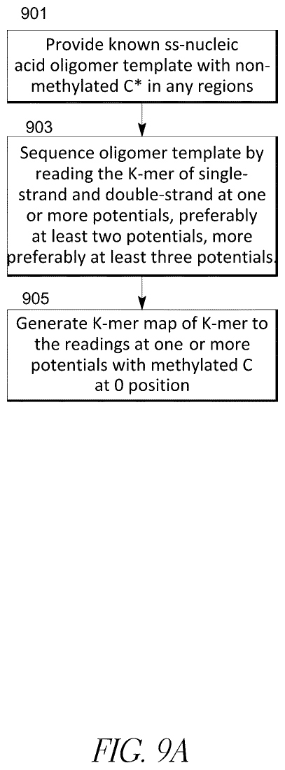

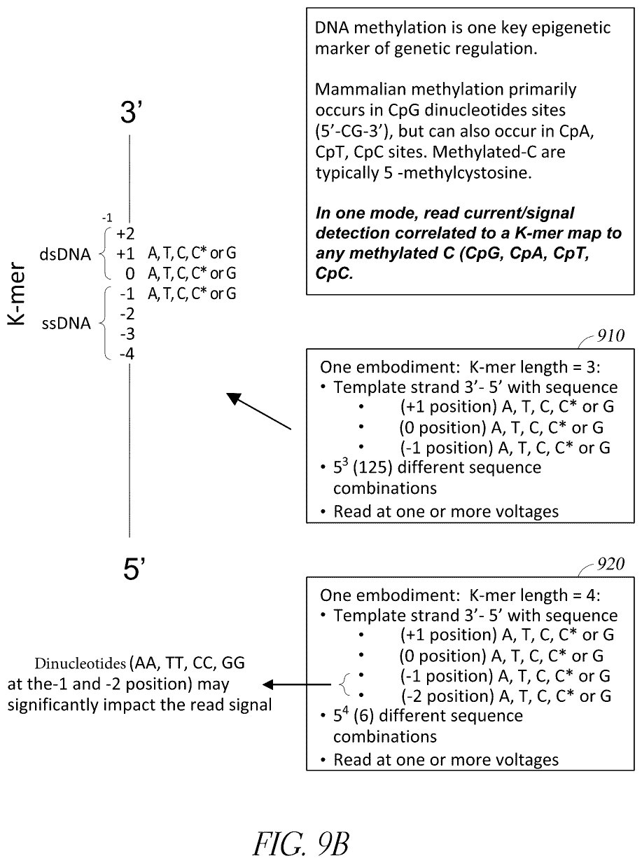

FIG. 9A illustrates a method of constructing a K-mer map for instances of K-mer including methylated C bases (e.g., CpG, CpA, CpT, or CpC), and using the K-mer map for sequencing. The method may start at block 901, providing known single stranded nucleic acid oligomer template with non-methylated C and methylated C* in any regions. The method may then move to block 903, sequencing oligomer template by reading the K-mer instances at one or more potential differences, preferably at least two potential differences, more preferably at least three potential differences. The method may then move to block 905, generating K-mer map of K-mer to the readings at one or more potential differences with methylated C at 0 position. Blocks 901 to 905 may represent steps performed by a sequencing instrument maker. The constructed K-mer map can then be provided for base calling while sequencing unknown ss-nuclei oligomer analyte template. In some embodiments, base calling may be performed by the instrument or a computer coupled to instrument with K-mer map loaded or stored on the instrument or computer. In some embodiments, base calling may be performed over the cloud (i.e., network) with K-map loaded on the cloud server.

FIG. 9B illustrates example K-mer instances and K-mer map constructions discussed in connection with FIG. 9A.

In certain embodiments, the K-mer map 910 length is a 3-mer map of three nucleotide positions comprising a single base position in the single-stranded region (i.e., position −1) of the template stand and two base positions of the double-stranded region (i.e., position +1 and position 0) of the template strand. The position +1 indicates the nucleotide of the template strand in which a nucleotide has been incorporated into the complementary strand in two incorporation steps prior to the current read step. The position 0 indicates the nucleotide of the template strand in which a nucleotide has been incorporated into the complementary strand in an incorporation step prior to the current read step. The position −1 indicates the nucleotide of the template strand one base position from position 0 towards the 5′ end of the template strand.

In certain embodiments, a 3-mer map, such as K-mer map 910, of three nucleotide positions comprises 125 possible states of five different nucleotides (e.g., A, T, C, methylated C*, or G) or 5^{K} states. In certain embodiments, a 3-mer map with a single read step at a single read voltage following an incorporate step comprises 125 signal entries for decoding the incorporated nucleotide at position 0. In certain embodiments, a 3-mer map with a multiple M read steps at multiple read voltages following an incorporate step comprises M times 5^{K} signal entries for decoding the incorporated nucleotide at position 0. For example, a 3-mer map with three read steps following an incorporate step comprises 3 times 5^{K} entries or 375 signal entries for decoding the incorporated nucleotide at position 0. Although a K-mer map may have a same signal reading for two different bases at a single read voltage, the likelihood decreases for have a same set of signal readings for two different incorporated bases as the number of read steps of different read voltages increases.

In certain embodiments, the K-mer map 920 length is a 4-mer map of four nucleotide positions comprising two base position in the single-stranded region (i.e., position −1 and position −2) of the template stand and two base positions of the double-stranded region (i.e., position +1 and position 0) of the template strand. The position +1 indicates the nucleotide of the template strand in which a nucleotide has been incorporated into the complementary strand in two incorporation steps prior to the current read step. The position 0 indicates the nucleotide of the template strand in which a nucleotide has been incorporated into the complementary strand in an incorporation step prior to the current read step. The position −1 indicates the nucleotide of the template strand one base position from position 0 towards the 5′ end of the template strand. The position −2 indicates the nucleotide of the template strand two base positions from position 0 towards the 5′ end of the template strand.

In certain embodiments, a 4-mer map, such as K-mer map 920, of four nucleotide positions comprises 625 possible states of five different nucleotides (e.g., A, T, C, methylated C*, or G) or 5^{K} states. In certain embodiments, a 4-mer map with a single read step at a single read voltage following an incorporate step comprises 625 signal entries for decoding the incorporated nucleotide at position 0. In certain embodiments, a 4-mer map with a multiple M read steps at multiple read voltages following an incorporate step comprises M times 5^{K} signal entries for decoding the incorporated nucleotide at position 0. For example, a 4-mer map with three read steps following an incorporate step comprises 3 times 5^{K} entries or 1,875 signal entries for decoding the incorporated nucleotide at position 0. Although a K-mer map may have a same signal reading for two different bases at a single read voltage, the likelihood decreases for have a same set of signal readings for two different incorporated bases as the number of read steps of different read voltages increases.

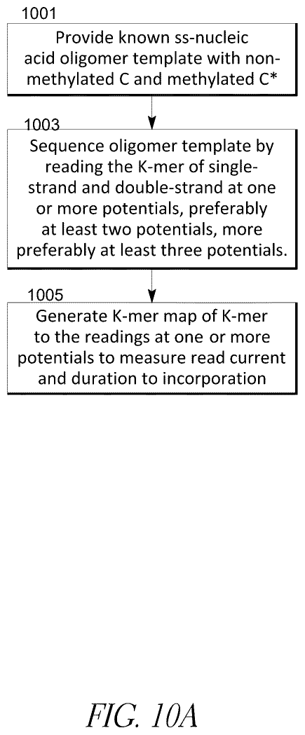

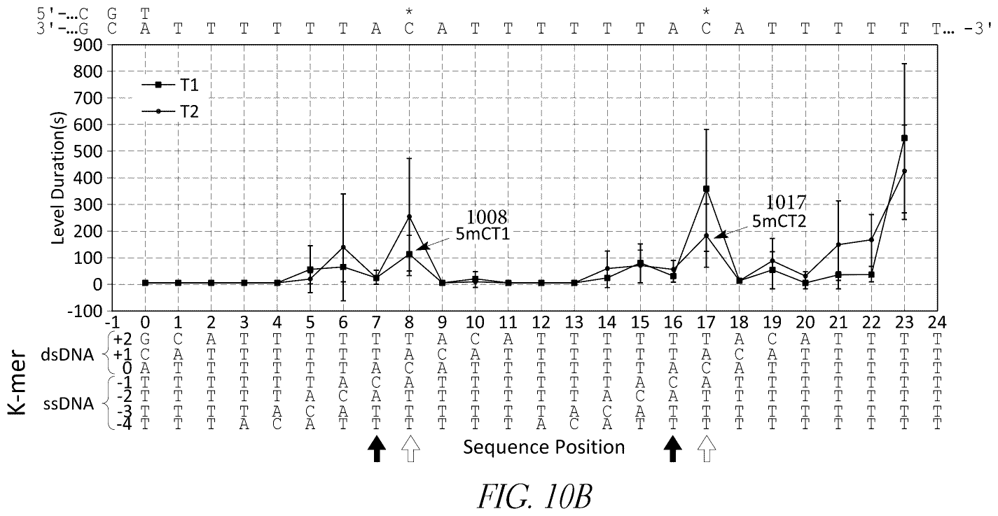

In certain embodiments of the K-mer maps, such as the K-mer maps as described in reference to FIGS. 9A-9B and FIGS. 10A-10B, the K-mer maps include position −1 and −2. Dinucleotides, such as AA, TT, CC, or GG, at position −1 and −2, provide a differentiated signal that increases the accuracy of decoding position 0 when decoding a nucleic acid region of various lengths.

FIG. 10A illustrates another method of constructing a K-mer map for instances of K-mer including methylated C bases (e.g., CpG, CpA, CpT, or CpC), and using the K-mer map for sequencing, where the base incorporation waiting time may be utilized. The method may start at block 1001, provide a known single stranded nucleic acid oligomer template with non-methylated C and methylated C*. The method may then move to block 1003, sequencing oligomer template by reading the K-mer instances at one or more potential differences, preferably at least two potential differences, more preferably at least three potential differences. The method may then move to block 1005, generating K-mer map of K-mer to the readings at one or more potential differences to measure read current and duration to incorporation (waiting time before a nucleotide is successfully incorporated). Blocks 1001 to 1005 may represent steps performed by a sequencing instrument maker. The constructed k-mer map can then be provided for base calling while sequencing unknown ss-nuclei oligomer analyte template. For example, base call a methyl C if the incorporation of a G is “relatively” quick. In some embodiments, base calling may be performed by the instrument or a computer coupled to instrument with K-mer map loaded or stored on the instrument or computer. In some embodiments, base calling may be performed over the cloud (i.e., network) with K-map loaded on the cloud server.

FIG. 10B shows experimental data relating to detecting epigenetic modifications via indirect kinetics, according to some embodiments of the disclosed technology. The level durations represent the waiting time before a nucleotide is successfully incorporated. The data show distinct and reproducible patterns between methylated and unmethylated bases. For example, base incorporation opposite the methylated C appears to be faster than opposite a natural C. Template T1 and Template T2 had identical sequences except Template T1 had a methylated C at sequence position 8 (see reference number 1008 in FIG. 10B) and a non-methylated C at sequence position 17, whereas Template T2 had a non-methylated C at sequence position 8 and a methylated C at sequence position 17 (see reference number 1017 in FIG. 10B). As can be seen from FIG. 10B, the incorporation time of Template T1 of a base at position 8 opposite a methylated C is relatively faster than the incorporation time of Template T2 of a base at position 8 opposite a non-methylated C. As can be seen from FIG. 10B, the incorporation time of Template T2 of a base at position 17 opposite a methylated C is relatively faster than the incorporation time of Template T1 of a base at position 17 opposite a non-methylated C.

Accordingly, disclosed methods of sequencing a template polynucleotide using a nanopore may include incorporating a nucleotide to extend a complementary polynucleotide; reading a pore current when a single-strand portion of the template polynucleotide and a duplex portion of the template polynucleotide and the complementary polynucleotide are held in a (non-translocating) position near the nanopore, e.g., within the nanopore such that extension of the complementary polynucleotide cannot occur; and comparing the pore current to a K-mer map, the K-mer map comprising a plurality of entries in which each entry includes a K-mer sequence and a pore current at an applied read potential, the K-mer sequence includes at least one position of the template polynucleotide in the single-strand portion and at least one position of the template polynucleotide in the duplex portion; and determining a sequence of the template polynucleotide based upon the comparison of the pore current to the K-mer map. In some embodiments, the pore current is measured under more than one potential or under multiple potentials. In some embodiments, the K-mer sequence of the K-mer map comprises at least two positions of the template polynucleotide in the single-strand portion. In some embodiments, the disclosed methods may further include determining a variation (e.g., coefficient of variation) of the read pore current, wherein the sequence of the template polynucleotide is further determined based upon the variation (e.g., coefficient of variation). In some alternative embodiments, the disclosed methods may determine a variation (e.g., a coefficient of variation) of the pore current and determine the sequence of the template polynucleotide based upon the variation (e.g., the coefficient of variation) alone. In some embodiments, the nucleotide is incorporated to extend the complementary polynucleotide when a 3′ end of the complementary polynucleotide is not sequestered within (e.g., positioned away from) the nanopore such that the 3′ end is placed in a location that is accessible to the polymerase and free nucleotides in the electrolyte solution for a set period of time. In some embodiments, the pore current is read when a 3′ end of the complementary polynucleotide is biased away from the nanopore (i.e., separated from the nanopore via the application of a voltage bias) for a set period of time. In some embodiments, the disclosed methods may further include identifying a methylated-C base in the determined sequency by an incorporation time of a nucleotide to extend the complementary polynucleotide.

### EXAMPLES

The following examples describe selected aspects of the sequencing embodiments described herein. The examples are intended for illustration purposes and should not be interpreted as limiting the scope of the claims unless explicitly recited in the claims.

### Example 1—Constructing a K-mer Map

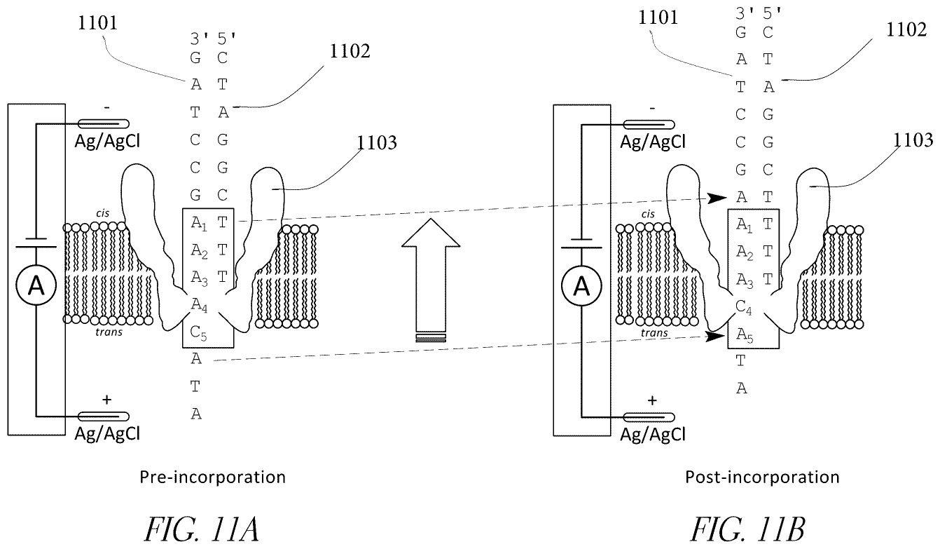

FIG. 11A, FIG. 11B and Table 1 illustrate an example of making a K-mer map having 5-mers. FIG. 11A illustrates a state of a target polynucleotide where the duplex region sensed by the nanopore recognition zone of the pore protein (1103) includes the 5-mer AAAAC in the template strand (1101) and the oligonucleotide TTT in the complementary strand (1102). FIG. 11B illustrates that the target polynucleotide has shifted with respect to the nanopore recognition zone of the pore protein (1103) after an additional nucleotide T was incorporated. Thus, in the new state, the duplex region sensed by the nanopore recognition zone includes the 5-mer AAACA in the template strand (1101) and the oligonucleotide TTT in the complementary strand (1102). For each 5-mer instance, the ionic current through the nanopore was measured at three different applied voltages, V1, V2 and V3, and the average value of the current is shown in Table 1. Thus, each 5-mer instance corresponds to a code that includes three values.

TABLE 1

| | Current (pA) | | |
|---|---|---|---|
| K-mer | V1 | V2 | V3 |
| ‘AAAAA’ | 46.191 | 41.449 | 37.194 |
| ‘AAAAC’ | 42.128 | 42.800 | 43.483 |
| ‘AAAAG’ | 48.527 | 42.307 | 36.884 |
| ‘AAAAT’ | 47.326 | 37.450 | 29.635 |
| ‘AAACA’ | 46.753 | 34.425 | 25.348 |
| ‘AAACC’ | 38.669 | 39.262 | 39.864 |
| ‘AAACG’ | 41.448 | 37.326 | 33.614 |

. . . Table continues to row 1,024 for 5-mer

### Example 2—K-mer Map States

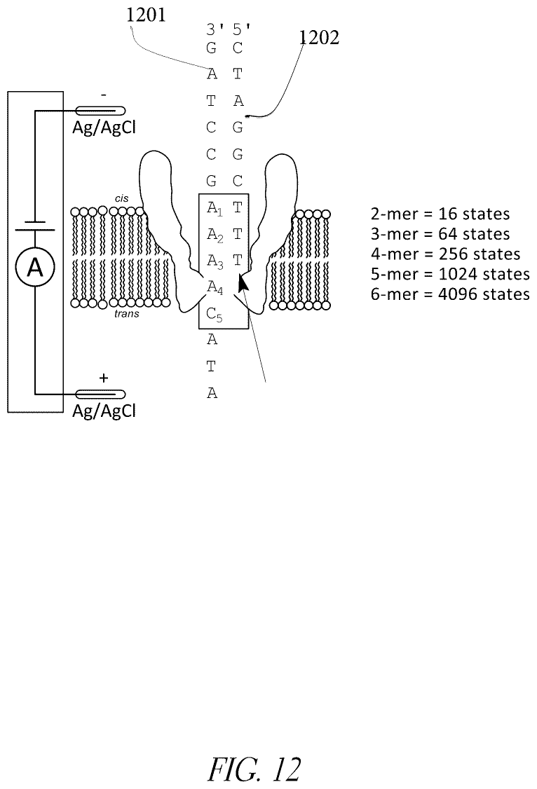

FIG. 12 illustrates examples of K-mer map states. If K positions contribute to the nanopore signal, mapping of 4^{K} possible sequence combinations (or states) are required to cover all possibilities. For example,
2-mer requires 16 states;
3-mer requires 64 states;
4-mer requires 256 states;
5-mer requires 1024 states;
6-mer requires 4096 states.

The complementary strand also affects the nanopore current. The nanopore current of the duplex of the template strand (1201) and the complementary strand (1202) in combination with the single strand of the template strand (1201) is measured in certain embodiments.

### Example 3.1—Base Calling Templates De Novo with a K-Mer Map

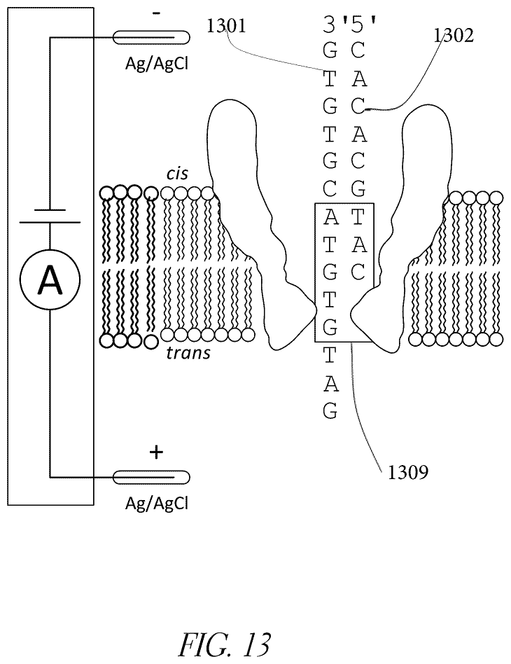

FIG. 13 and Table 2 illustrate an example of de novo sequencing of template polynucleotides according to some embodiments of the disclosed technology. FIG. 13 illustrates that a K-mer (1309) that is 5 nucleotides long is used, which includes 3 bases in the double-strand region at positions +2, +1, and 0 and 2 bases in the single-strange region at positions −1 and −2. A bijective map (see Table 2 for an example) was generated compiling all sequence-specific information for each read state for the K-mers. In one embodiment, the template strand (1301) can be decoded by determining the state of the template strand which best fits the read step(s), such as three read steps as shown in Table 2. Decoding of a template strand can be done by analyzing the read levels to determine the identity of the incorporated base in the complementary strand (1302) at position 0 to determine the identity of the template strand at position 0. Each incorporated base can be analyzed individually in reference to the K-mer map in which each cycle of the incorporated base is analyzed without reference to each other.

In Table 2, row 1, columns 1 and 2 represent portions of the sequence of the analyte template DNA strand (Level ID 0) with a dsDNA portion and a ssDNA portion held in a position within the nanopore (such that DNA synthesis cannot occur) at the start of sequencing. In other words, the “dsDNA” portion is a portion of the analyte template DNA that forms a duplex with the daughter strand. Three current reads at three different applied voltages (row 1, last three columns) are matched to one of the 5-mers of the K-mer map, and the 5-mer region of the template DNA strand affecting the nanopore current is determined to be the State GCATG. The 5-mer affecting the nanopore current includes 3 bases in the dsDNA portion and 2 bases of the ssDNA portion.

Row 2, columns 1 and 2 represent portions of the sequence of the analyte template DNA strand (Level ID 1) after incorporation of a base opposite the first “T” base of the ssDNA portion of Level ID 0 (row 1, column 2). Three current reads at three different applied voltages (row 2, last three columns) are matched to one of the 5-mers of the K-mer map, and the 5-mer region of the template DNA strand affecting the nanopore current is determined to be the State CATGT.

Row 3, columns 1 and 2 represent portions of the sequence of the analyte template DNA strand (Level ID 2) after incorporation of a base opposite the first “G” base of the ssDNA portion of Level ID 1 (row 2, column 2). Three current reads at three different applied voltages (row 3, last three columns) are matched to one of the 5-mers of the K-mer map, and the 5-mer region of the template DNA strand affecting the nanopore current is determined to be the State ATGTG.

TABLE 2
5-mer state map

| | | | Le- | State | | | |
|---|---|---|---|---|---|---|---|
| | | | vel | | | | |
| dsDNA | SSDNA | State | ID | ID | 40mV | 60mV | 80mV |
| TCGCA | TGTGT | GCATG | 0 | 0 | 4.385 | 8.812 | 15.314 |
| CGCAT | GTGTG | CATGT | 1 | 1 | 3.184 | 6.088 | 10.377 |
| GCATG | TGTGA | ATGTG | 2 | 2 | 5.545 | 10.038 | 15.86 |
| CATGT | GTGAA | TGTGT | 3 | 3 | 3.728 | 6.216 | 9.881 |
| ATGTG | TGAAT | GTGTG | 4 | 9 | 5.493 | 10.045 | 15.819 |
| TGTGT | GAATG | TGTGA | 5 | 79 | 5.069 | 8.285 | 12.548 |
| GTGTG | AATGT | GTGAA | 6 | 80 | 6.062 | 11.865 | 18.575 |
| TGTGA | ATGTG | TGAAT | 7 | 81 | 3.994 | 8.321 | 15.511 |
| GTGAA | TGTGT | GAATG | 8 | 82 | 4.004 | 8.18 | 14.512 |
| TGAAT | GTGTA | AATGT | 9 | 14 | 3.71 | 7.606 | 13.066 |
| GAATG | TGTAC | ATGTG | 10 | 2 | 5.545 | 10.038 | 15.86 |
| AATGT | GTACG | TGTGT | 11 | 3 | 3.728 | 6.216 | 9.881 |

### Example 3.2—Base Calling Templates de novo with a HMM

Another example of de novo sequencing of template polynucleotides according to some embodiments of the disclosed technology is described below, which is particularly useful when it is difficult to find a bijective mapping between the polynucleotide sequence interacting with the nanopore read head and the associated nanopore ionic current. For example, the value of the mean nanopore current generated by a given polynucleotide sequence under a certain applied voltage may exhibit a degree of randomness. In certain embodiments, a Hidden Markov Model (HMM) is used to describe the probabilistic mapping between the polynucleotide sequence interacting with the nanopore readhead and the associated nanopore ionic current, and the Viterbi algorithm or other decoding models is used to determine the most probable sequence of states of the template strand based upon a cycle of read step(s), such as three read steps at three different applied voltages, after multiple incorporations of bases into the complementary strand to explain the observed nanopore signals. The HMM was built on a Markov chain describing the possible transitions among a set of K-mer states and the possible associated nanopore signals as the experimental observables. In some embodiments, only the mean and noise of the nanopore currents are extracted from the ionic current trace and are used to represent the experimental observables. In other embodiments, only the mean of the nanopore currents is extracted from the ionic current trace and is used to represent the experimental observable. The HMM has a set of transition probabilities associated with the transitions among the K-mer states, and a set of emission probabilities that determine how the observed nanopore signals will be distributed given the K-mer states.

The unknown transition and emission probabilities of the HMM can be obtained by training the HMM with an expectation-maximization algorithm, such as the Baum-Welch algorithm. The training data set are obtained by sequencing and recording the measured nanopore signals according to the methods described herein with a known de Bruijn sequence containing, for example, all 4^{K} K-mer states (consistent with the definition illustrated in FIG. 13) that interact with the nanopore readhead. In other embodiments, the de Bruijn sequence containing 5^{K} K-mer states or 6^{K} K-mer states may be used, if the polynucleotide is formed of more than the 4 types of canonical bases (A, T, C, and G), such as when DNA or RNA modifications or non-natural bases are present in the polynucleotide.

After obtaining the transition and emission probabilities of the HMM by training the HMM with the training data set, the Viterbi algorithm can be used to decode/solve for the template polynucleotide sequence based on the measured nanopore signals. The Viterbi algorithm can determine the sequence path that best describes the observed nanopore signal data for a template polynucleotide of unknown nucleotide sequence, i.e., the most likely series of K-mer states (allowing only state transitions that are physically possible for a polynucleotide sequence) that underlies an observed series of nanopore signals (measured over a series of nucleotide incorporation operations according to the methods described herein). Although the K-mer state has a certain length, such as a 5-mer state, the HMM and Viterbi algorithm can use the information of multiple incorporations to determine the likely state of the template strand beyond just the number of base positions, such as beyond the five base positions of the 5-mer state. In other words, a decoding model (such as a HMM and Viterbi algorithm, analyzing each incorporated base collectively in reference to the K-mer states to determine which overall sequence of the template strand is the most likely to fit the collective reads of a cycle of incorporated bases.

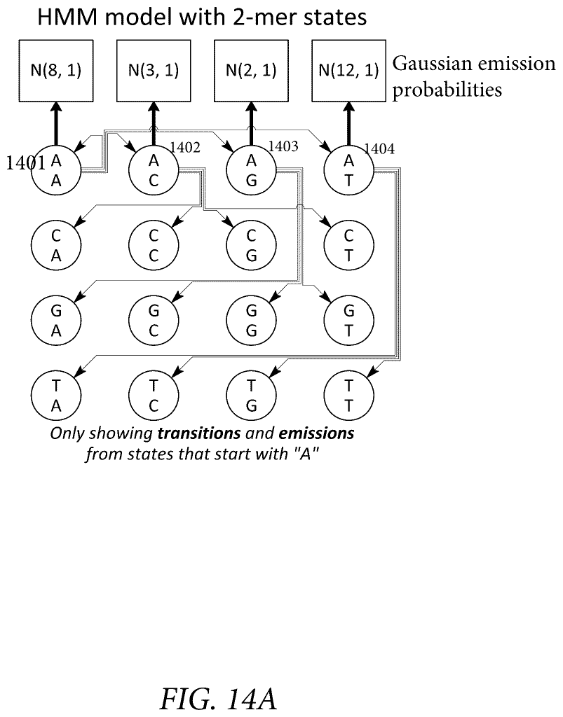

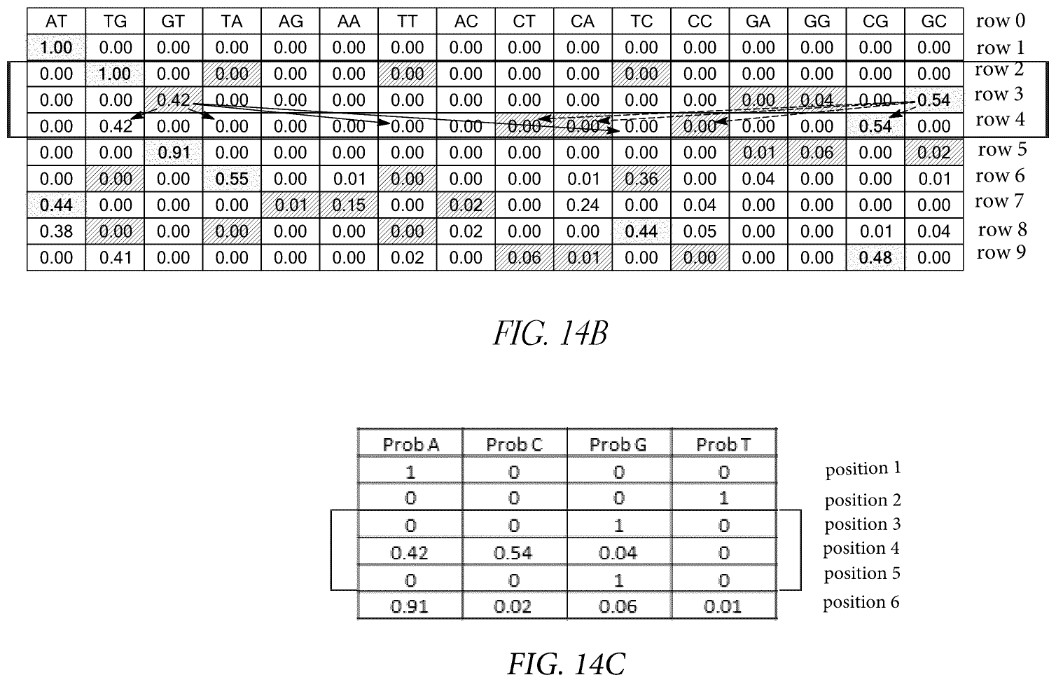

As an example, a simplified HMM with 2-mer states is shown in FIG. 14A for illustration purposes (assuming that the region of the polynucleotide interacting with the nanopore readhead is a 2-mer). Also, only transitions (arrows with thin lines) and emissions (arrows with thick lines) from states that start with “A” (1401, 1402, 1403, 1404) are shown. The simplified HMM in FIG. 14A has a set of start probabilities where the first 2-mer of the template strand starts with probability=1.0, a set of transition probabilities where all state transition probabilities=0.25, and a set of emission probabilities that are assumed to take Gaussian distributions. The Gaussian distributions determine the possible observed mean nanopore currents. For example, the emission probability for the state “AA” is a normal distribution N(8, 1), having a mean=8 and a variance=1. Therefore, the mean nanopore current, when “AA” is interacting with the nanopore readhead, is assumed to be normally distributed with mean=8 (current unit) and standard deviation=1 (current unit).

Row 1 to row 9 of FIG. 14B show, given a series of 9 observed nanopore signals, the posterior probability that the region of the polynucleotide interacting with the nanopore readhead, during each of the 9 observations, is in any of the 2-mer states listed in row 0. Since the transition and emission probabilities are known, the most probable polynucleotide sequence underlying the given series of observations can be decoded by the Viterbi algorithm, and these posterior probabilities in FIG. 14B are computed during the decoding process. Furthermore, the probability of the nucleotide at each position along the template polynucleotide having an identity of A, T, C or G is computed and shown in FIG. 14C. Based on the information shown in FIG. 14B and FIG. 14C, the probability that the nucleotide at a position along the template polynucleotide has a certain identity, given the series of observed nanopore signals and that the nucleotide at the previous position is in the highest probability state for that position, can be computed and used to determine the polynucleotide sequence.

Further details regarding the Hidden Markov Model may be found in “Yoon, B. J., 2009. Hidden Markov models and their applications in biological sequence analysis. Current genomics, 10(6), pp. 402-415” and U.S. patent Ser. No. 11/049,588, the disclosures of each of which is incorporated herein by reference. In other embodiments, any suitable machine learning models can be used to determine a polynucleotide sequence. For example, the machine learning model may be a classification machine learning model.

In some cases, as discussed above in connection with FIG. 16A, FIG. 16B, FIG. 16C and FIG. 16D, the system exhibits voltage-dependent modulation of the polynucleotide-nanopore interaction and non-Ohmic behavior of the ionic current. For example, the situations shown in FIG. 16A, FIG. 16B, and FIG. 16C has the same “level number” (the number of nucleotides that has been incorporated into the complementary strand after a series of incorporation operations), but the definition of the K-mer differs between the situation of FIG. 16A and the situation of FIG. 16B (being “TATTT” and “ATATT”, respectively). Therefore, in some embodiments, a separate HMM with its own K-mer definition may be constructed for each situation of a different applied voltage and trained separately. Decoding for an unknown polynucleotide sequence may be performed by separately decoding in each situation of a different applied voltage using the different HMMs, and then combining these results.

FIG. 17A shows the same situation as in FIG. 16A. FIG. 17B illustrates that when the level number increases by 1, and the applied voltage is 60 mV, the portion “TATTT” in the template strand 1701 and the portion “ATA” in the complementary strand 1702 (indicated by the dashed boxes) are the regions of the polynucleotide that affect the measured ionic current trace. Therefore, the same K-mer definition (“TATTT”) can be used for the situation of FIG. 17A and the situation of FIG. 17B. Thus, in some embodiments, only one HMM is constructed in which one K-mer state definition can be associated with different level numbers under different applied voltages. Decoding for an unknown polynucleotide sequence may be performed by utilizing only one HMM.

### Example 4—Utilize Duplex Read Noise

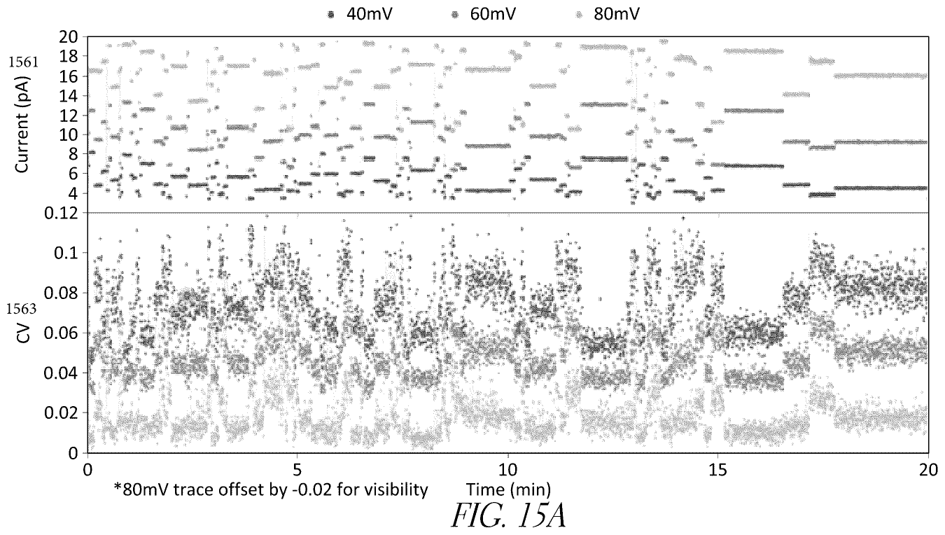

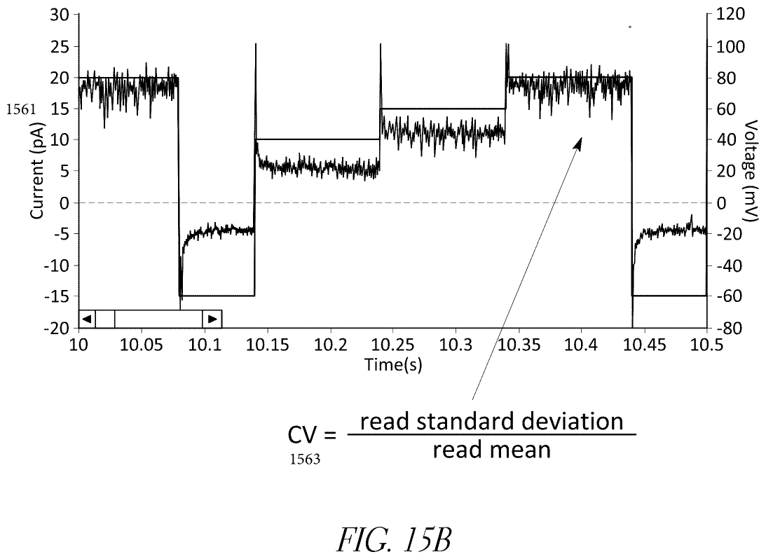

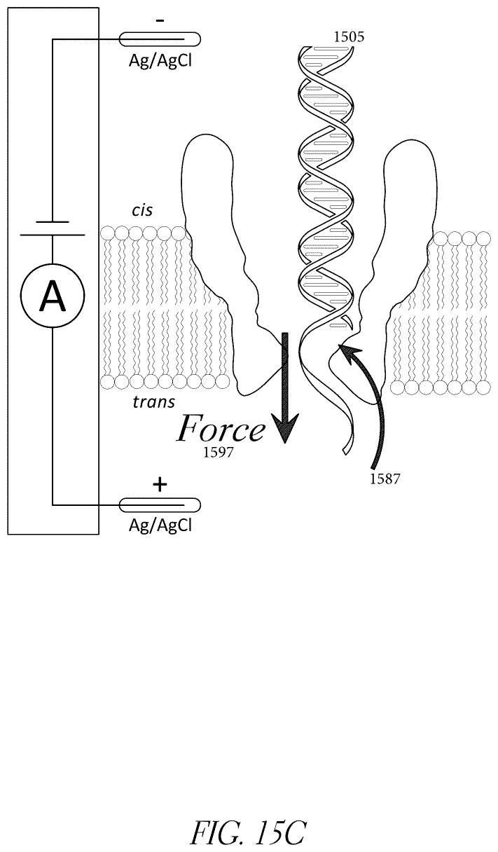

FIG. 15A, FIG. 15B and FIG. 15C show an example of using noise patterns of nanopore signals. The nanopore current of the duplex of the template strand and the complementary strand in combination with the single strand of the template strand is measured in certain embodiments.

FIG. 15A shows experimental results of the mean nanopore ionic current, 1561, (details are similar to those explained with FIG. 6) and the coefficient of variation (CV), 1563, of the measured pore current, measured at three different applied voltages. Both the mean current and the CV exhibit discrete changes upon incorporation of an additional nucleotide. Both the mean current and the CV exhibit distinct, reproducible, sequence-dependent results as the same polynucleotide is sequenced more than once. As demonstrated by the experimental results, noise pattern of the nanopore signal also contains the polynucleotide sequence information. For example, a high CV is generally correlated with an A base. Therefore, in addition to the mean current, a noise pattern of the nanopore signal can be used in decoding polynucleotide sequences. For example, each K-mer instance can correspond to a code that includes six values (mean current and CV at three applied voltages).

FIG. 15B illustrates how CV 1563 is extracted from the measured ionic current 1561 through nanopore as a function of time. The CV is defined as the standard deviation divided by mean current for a read as defined with FIG. 2B. In other embodiments, other indictors to determine the sequence of the template strand may be determined and may be used from the noise pattern.

FIG. 15C illustrates that, without being bound by theory, duplex “peeling” (1587) under force may contribute to the sequence-specific noise response. The force (1597) applied to the duplex (1505) is selected based upon the applied voltage at each read step. The noise pattern of the nanopore current is determined by the sequence of the duplex due to the different bases within the nanopore, such as the different bases of the duplex within the nanopore and/or the different bases of the single stranded template within the nanopore.

### Example 5—Sequencing PhiX Bacteriophage Genome

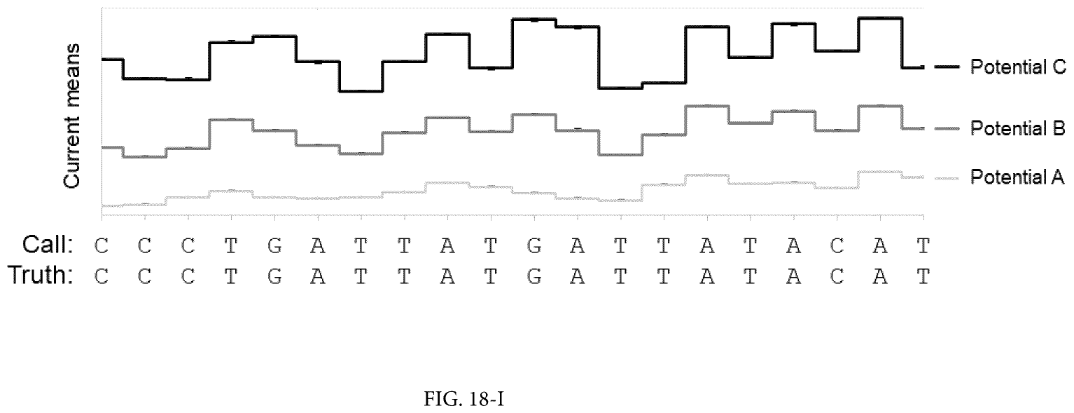

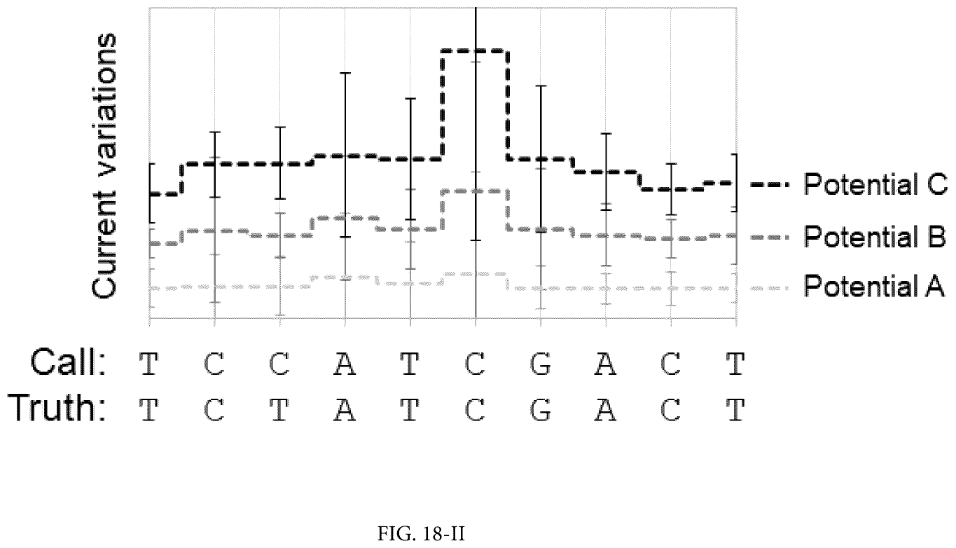

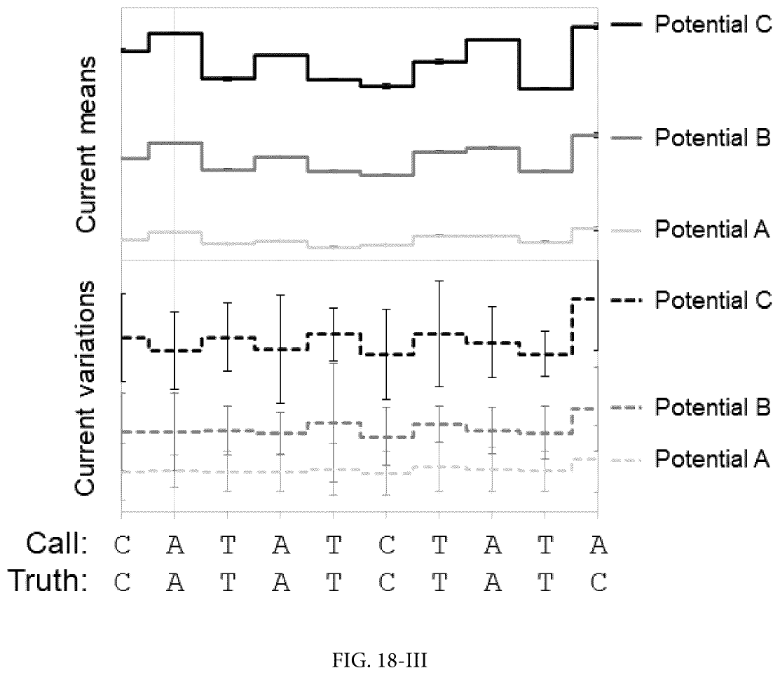

FIG. 18-I shows results of calling bases with current means only. De novo sequencing of 20 nucleotides was conducted on a template polynucleotide containing a region 1 of the PhiX bacteriophage genome with the sequence CCCTGATTATGATTATACAT. Nanopore current means were recorded at a series of applied potentials when a single-stranded portion of the template polynucleotide and a duplexed portion of the template polynucleotide and the complementary polynucleotide were held in a position within the nanopore such that extension of the complementary polynucleotide cannot occur. The called polynucleotide sequence using current means at multiple applied potentials was CCCTGATTATGATTATACAT. Table 3-I shows that when using current means from a single applied potential, 9 basecalling errors were obtained. When using current means from several applied potentials, 0 basecalling errors were obtained.

TABLE 3-I
PhiX Region 1 (20 nt)

| Decoding Configuration | Errors |
|---|---|
| Current means only, Single potential | 9 |
| Current means only, Multiple potentials | 0 |

FIG. 18-II shows results of calling bases with current variations only. De novo sequencing of 10 nucleotides was conducted on a template polynucleotide containing a region 2 of the PhiX bacteriophage genome with the sequence TCTATCGACT. Nanopore current variations were recorded at a series of applied potentials when a single-stranded portion of the template polynucleotide and a duplexed portion of the template polynucleotide and the complementary polynucleotide were held in a position within the nanopore such that extension of the complementary polynucleotide cannot occur. The called polynucleotide sequence using current variations at multiple applied potentials was TCCATCGACT. Table 3-II shows that when using current variations from a single applied potential, 5 basecalling errors were obtained. When using current variations from several applied potentials, 1 basecalling error was obtained.

TABLE 3-II
PhiX Region 2 (10 nt)

| Decoding Configuration | Errors |
|---|---|
| Current variations only, Single potential | 5 |
| Current variations only, Multiple potentials | 1 |

FIG. 18-III shows results of calling bases with current means and variations. De novo sequencing of 10 nucleotides was conducted on a template polynucleotide containing a region 3 of the PhiX bacteriophage genome with the sequence CATATCTATC. Nanopore current means and current variations were recorded at a series of applied potentials when a single-stranded portion of the template polynucleotide and a duplexed portion of the template polynucleotide and the complementary polynucleotide were held in a position within the nanopore such that extension of the complementary polynucleotide cannot occur. The called polynucleotide sequence using both current mean and current variation measurements at multiple applied potentials was CATATCTATA. Table 3-III shows that when using current means from several applied potentials but not current variations, 5 basecalling errors were obtained. When using current variations from several applied potentials but not current means, 6 basecalling errors were obtained. When using both current means and current variations from several applied potentials, 1 basecalling error was obtained.

TABLE 3-III
PhiX Region 3 (10 nt)

| Decoding Configuration | Errors |
|---|---|
| Current means only, Multiple potentials | 5 |
| Current variations only, Multiple potentials | 6 |
| Current means and variations, Multiple potentials | 1 |

### Additional Notes

It should be understood that the data or points in the K-mer map suitably may be stored any suitable format, within a non-volatile computer-readable medium, that correlates a given measurement condition with one or more values that were measured under that condition and combinations of duplex and single-stranded sequences that were used to generate those values. A look-up table (LUT) is a non-limiting example of a format that may be used to store correlations between measured values and known combinations of duplex and single-stranded sequences that were used to generate those values. However, it will be understood that any suitable data structure may be used to store correlations between measured values and known combinations of duplex and single-stranded sequences that were used to generate those values. For example, the data structure may be generated by, and appropriately stored for use by, a machine learning algorithm. For example, the data structure may be generated by training a machine learning algorithm to recognize values that are obtained, under each respective given set of measurement conditions, and are known a priori to correspond to respective combinations of duplex and single-stranded sequences, and may be stored in any suitable format within a non-volatile computer-readable medium. The data structure subsequently may be used by the trained machine learning algorithm to generate an output that identifies nucleotides in the sequence of polynucleotide, based upon an input of values that are measured in between nucleotide addition steps. In certain examples, the data structure may be generated by, and appropriately stored for use by, a neural network, such as a deep learning algorithm. For example, the data structure may be generated by training a neural network (e.g., deep learning algorithm) to recognize values that are obtained, under each respective set of measurement conditions, and are known a priori to correspond to respective combinations of duplex and single-stranded sequences, and may be stored in any suitable format within a non-volatile computer-readable medium. Accordingly, the data structure may include neurons of the neural network (e.g., deep learning algorithm). The data structure subsequently may be used by the trained neural network (e.g., deep learning algorithm) to generate an output that identifies nucleotides in the sequence of polynucleotide, based upon an input of values that are measured in between nucleotide addition steps.

The data structure may be generated by training any suitable machine learning algorithm, such as a neural network (e.g., deep learning algorithm) using measured values, the combinations of nucleotides where are known a priori to correspond to those measured values, and the measurement conditions under which those measured values were obtained. In this regard, data structure may have a construction that is readily usable by the trained machine learning algorithm, e.g., trained neural network, such as a trained deep learning algorithm to identify combinations of nucleotides using measured values, but such construction may not necessarily be usable by any other software, module, or algorithm to determine correlations between measured values and unknown combinations of nucleotides. For example, machine learning algorithms (such as neural networks, e.g., deep learning algorithms) may be trained, using the measured signal, to make base calls. Non-limiting examples of machine learning algorithms are supervised, semi-supervised, unsupervised, and reinforcement algorithms. Neural network algorithms are a subset of machine learning algorithms and may include deep learning algorithms, convolutional neural networks, recurrent neural networks, generative adversarial networks, and recursive neural networks. Accordingly, the particular construction of data structure may include, for example, a vector space, graph space, neurons of a neural network, or the like. Alternatively, data structure may be implemented using any suitable data structure that may be queried using nucleotide identification module, such as a look-up table (LUT), matrix, flat-file database structure, SQL database structure, or the like.

It should be appreciated that the controller or processor may be implemented using any suitable combination of digital electronic circuitry, integrated circuitry, application specific integrated circuits (ASICs), field programmable gate arrays (FPGAs), central processing units (CPUs), graphical processing units (GPUs), computer hardware, firmware, software, and/or combinations thereof. For example, one or more functionalities of the controller or processor may be implemented in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system can include clients and servers. A client and server are generally remote from each other and typically interact through a communication network. The relationship of client and server arises by virtue of computer programs running on the respective computers and having a client-server relationship to each other.

These computer programs, which can also be referred to as modules, programs, software, software applications, applications, components, or code, can include machine instructions for a programmable processor, and/or can be implemented in a high-level procedural language, an object-oriented programming language, a functional programming language, a logical programming language, and/or in assembly/machine language. As used herein, the terms “memory” and “computer-readable medium” refer to any computer program product, apparatus and/or device, such as magnetic discs, optical disks, solid-state storage devices, memory, and Programmable Logic Devices (PLDs), used to provide machine instructions and/or data to a programmable data processor, including a machine-readable medium that receives machine instructions as a computer-readable signal. The term “computer-readable signal” refers to any signal used to provide machine instructions and/or data to a programmable data processor. The computer-readable medium can store such machine instructions non-transitorily, such as would a non-transient solid-state memory or a magnetic hard drive or any equivalent storage medium. The computer-readable medium can alternatively or additionally store such machine instructions in a transient manner, such as for example as would a processor cache or other random access memory associated with one or more physical processor cores.

The computer components, software modules, functions, data stores and data structures described herein can be connected directly or indirectly to each other in order to allow the flow of data needed for their operations. It is also noted that a module or processor includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality can be located on a single computer or distributed across multiple computers and/or the cloud, depending upon the situation at hand.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 nm to about 20 nm should be interpreted to include not only the explicitly recited limits of from about 2 nm to about 20 nm, but also to include individual values, such as about 3.5 nm, about 8 nm, about 18.2 nm, etc., and sub-ranges, such as from about 5 nm to about 10 nm, etc. Furthermore, when “about” and/or “substantially” are/is utilized to describe a value, this is meant to encompass minor variations (up to +/−10%) from the stated value.

While several examples have been described in detail, it is to be understood that the disclosed examples may be modified. Therefore, the foregoing description is to be considered non-limiting.

While certain examples have been described, these examples have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Example polynucleotide pluralities include, for example, populations of about 1×105 or more, 5×105 or more, or 1×106 or more different polynucleotides. Accordingly, the definition of the term is intended to include all integer values greater than two. An upper limit of a plurality may be set, for example, by the theoretical diversity of polynucleotide sequences in a sample.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A method of sequencing a template polynucleotide using a nanopore, comprising:
incorporating a nucleotide to extend a complementary polynucleotide;
reading a pore current at an applied read potential when a single-stranded portion of the template polynucleotide and a duplex portion of the template polynucleotide and the complementary polynucleotide are held in a position within the nanopore such that extension of the complementary polynucleotide cannot occur;
comparing the pore current to a K-mer map, the K-mer map comprising a plurality of entries in which each entry includes a K-mer sequence and a pore current at the applied read potential, the K-mer sequence includes at least one position of the template polynucleotide in the single-strand portion and at least one position of the template polynucleotide in the duplex portion; and
determining a sequence of the template polynucleotide based upon the comparison of the pore current to the K-mer map.

2. The method of claim 1, wherein the pore current is measured at more than one potential.

3. The method of any of the claims 1-2, wherein the K-mer sequence of the K-mer map comprises at least two positions of the template polynucleotide in the single-stranded portion.

4. The method of any of the claims 1-3, further comprising determining a variation of the read pore current, wherein the sequence of the template polynucleotide is further determined based upon the variation.

5. The method of any of claims 1-4, further comprising identifying an epigenetically modified base in the determined sequence by an altered current relative to the non-modified base and/or an incorporation time of a nucleotide to extend the complementary polynucleotide.

6. The method of any of claims 1-5, wherein the nucleotide is incorporated to extend the complementary polynucleotide when the 3′ end of the complementary polynucleotide is not sequestered within the nanopore.

7. The method of any of claims 1-6, wherein the pore current is read when the 3′ end of the complementary polynucleotide is positioned within the nanopore constriction zone.

8. A method of sequencing a template polynucleotide using a nanopore, comprising:
incorporating a nucleotide to extend a complementary polynucleotide;
reading a first pore current at a first applied read potential when a single-stranded portion of the template polynucleotide and a duplex portion of the template polynucleotide and a complementary polynucleotide are held in a position near the nanopore such that extension of the complementary polynucleotide cannot occur;
reading a second pore current at a second applied read potential when the single-stranded portion of the template polynucleotide and the duplex portion of the template polynucleotide and the complementary polynucleotide are held in a position within the nanopore such that extension of the complementary polynucleotide cannot occur;
comparing the first pore current and the second pore current to a K-mer map, the K-mer map comprising a plurality of entries in which each entry includes a K-mer sequence and a plurality of pore currents at a plurality of applied read potentials, the K-mer sequence includes at least one position of the template polynucleotide in the single-stranded portion and at least one position of the template polynucleotide in the duplex portion; and
determining a sequence of the template polynucleotide based upon the comparison of the pore current to the K-mer map.

9. The method of claim 8, wherein the K-mer sequence of the K-mer map comprises at least two positions of the template polynucleotide in the single-stranded portion.

10. The method of any of the claims 8-9, further comprising determining a variation of the read pore current, wherein the sequence of the template polynucleotide is further determined based upon the variation.

11. The method of any of claims 8-10, further comprising identifying an epigenetically modified base in the determined sequence by an altered current relative to the non-modified base and/or an incorporation time of a nucleotide to extend the complementary polynucleotide.

12. The method of any of claims 8-11, wherein the nucleotide is incorporated to extend the complementary polynucleotide when the 3′ end of the complementary polynucleotide is not sequestered within the nanopore.

13. The method of any of claims 8-12, wherein the pore current is read when the 3′ end of the complementary polynucleotide is positioned within the nanopore constriction zone.

14. A method of sequencing a template polynucleotide using a nanopore, comprising:
performing a plurality of cycles, each cycle comprising
incorporating a nucleotide to extend the complementary polynucleotide to the template polynucleotide; and
reading a pore current of a single-stranded portion of the template polynucleotide and a duplex portion of the template polynucleotide and the complementary polynucleotide held in a position within the nanopore such that extension of the complementary polynucleotide cannot occur;
removing the complementary polynucleotide from the template polynucleotide after one set of the plurality of cycles; and
performing a second plurality of cycles and determining a second sequence of the template polynucleotide from the pore current reads of the second plurality of cycles.

15. The method of claim 14, wherein determining the sequence comprises comparing the current trace to a K-mer map, the K-mer map comprising a plurality of entries in which each entry includes a K-mer sequence and a pore current at an applied read potential, the K-mer sequence includes at least one position of the template polynucleotide in the single-stranded portion and at least one position of the template polynucleotide in the duplex portion.

16. The method of any of the claims 14-15, wherein the pore current is measured at more than one potential.

17. The method of any of the claims 14-16, wherein the K-mer sequence of the K-mer map comprises at least two positions of the template polynucleotide in the single-stranded portion.

18. The method of any of the claims 14-17, further comprising determining a variation of the read pore current, wherein the sequence of the template polynucleotide is further determined based upon the variation.

19. The method of any of claims 14-18, further comprising identifying an epigenetically modified base in the determined sequence by an altered current relative to the non-modified base and/or an incorporation time of a nucleotide to extend the complementary polynucleotide.

20. The method of any of claims 14-19, wherein the nucleotide is incorporated to extend the complementary polynucleotide when the 3′ end of the complementary polynucleotide is not sequestered within the nanopore.

21. The method of any of claims 14-20, wherein the pore current is read when the 3′ end of the complementary polynucleotide is positioned within the nanopore constriction zone.

22. A system for identifying a base sequence of a template polynucleotide, the system comprising:
an instrument comprising:
a nanopore in contact with a solution; and
a processor configured to selectively move the template polynucleotide relative to nanopore and configured to measure a pore current of a single-stranded portion of the template polynucleotide and a duplex portion of the template polynucleotide and the complementary polynucleotide; and
a non-volatile data storage medium coupled to the instrument, wherein the non-volatile data storage medium stores a K-mer map, the K-mer map comprising a plurality of entries in which each entry includes a K-mer sequence and a pore current at an applied read potential, the K-mer sequence includes at least one position of the template polynucleotide in the single-stranded portion and at least one position of the template polynucleotide in the duplex portion.

23. The system of claim 22, wherein instrument comprises the non-volatile data storage medium.

24. The system of claim 23, wherein the processor is further configured to determine the base sequence of the template polynucleotide based on the measured pore current and the K-mer map.

25. The system of claim 22, further comprising a computer attached locally to the instrument, wherein the computer comprises the non-volatile data storage medium.

26. The system of claim 22, further comprising a computer networked to the instrument, wherein the computer comprises the non-volatile data storage medium.

27. The system of claim 25 or 26, further comprising a computer attached locally to the instrument, wherein the computer is further configured to determine the base sequence of the template polynucleotide based on the measured pore current and the K-mer map.

28. A method of sequencing a template polynucleotide using a nanopore, comprising:
performing a plurality of cycles, each cycle comprising
incorporating a nucleotide to extend a complementary polynucleotide;
reading a pore current when a single-stranded portion of the template polynucleotide and a duplex portion of the template polynucleotide and the complementary polynucleotide are held in a position within the nanopore such that extension of the complementary polynucleotide cannot occur; and
determining a most probable sequence of the template polynucleotide based upon applying a trained statistical model to a series of pore currents respectively read in the plurality of cycles.

29. The method of claim 28, wherein the pore current in at least some of the plurality of cycles is measured at more than one potential.

30. The method of any of the claims 28-29, wherein the single-stranded portion comprises at least two positions of the template polynucleotide.

31. The method of any of the claims 28-30, wherein the statistical model is a Hidden Markov model.

32. The method of any of claims 28-31, further comprising identifying an epigenetically modified base in the determined sequence by an altered current relative to the non-modified base and/or an incorporation time of a nucleotide to extend the complementary polynucleotide.

33. The method of any of claims 28-32, wherein the nucleotide is incorporated to extend the complementary polynucleotide when the 3′ end of the complementary polynucleotide is not sequestered within the nanopore.

34. The method of any of claims 28-33, wherein the pore current in each cycle is read when the 3′ end of the complementary polynucleotide is positioned within the nanopore constriction zone.

35. A method of sequencing a template polynucleotide using a nanopore, comprising:
incorporating a nucleotide to extend a complementary polynucleotide;
determining a variation of a pore current when a single-stranded portion of the template polynucleotide and a duplex portion of the template polynucleotide and the complementary polynucleotide are held at an applied read potential in a position within the nanopore such that extension of the complementary polynucleotide cannot occur;
comparing the variation of the pore current to a K-mer map, the K-mer map comprising a plurality of entries in which each entry includes a K-mer sequence and a pore current variation at the applied read potential, the K-mer sequence includes at least one position of the template polynucleotide in the single-stranded portion and at least one position of the template polynucleotide in the duplex portion; and
determining a sequence of the template polynucleotide based upon the comparison of the variation of the pore current to the K-mer map.

36. The method of claim 35, wherein the pore current is measured at more than one potential and the variation of the pore current is determined at more than one potential.

37. The method of any of the claims 35-36, wherein the K-mer sequence of the K-mer map comprises at least two positions of the template polynucleotide in the single-stranded portion.

38. The method of any of claims 35-37, further comprising identifying an epigenetically modified base in the determined sequence by an altered pore current variation relative to the non-modified base and/or an incorporation time of a nucleotide to extend the complementary polynucleotide.

39. The method of any of claims 35-38, wherein the nucleotide is incorporated to extend the complementary polynucleotide when the 3′ end of the complementary polynucleotide is not sequestered within the nanopore.

40. The method of any of claims 35-39, wherein the variation of the pore current is determined when the 3′ end of the complementary polynucleotide is positioned within the nanopore constriction zone.
